(19) United States
(12) Patent Application Publication
(10) Pub. No.: US 2023/0159896 A1
(43) Pub. Date: May 25, 2023

(54) MULTI-LINEAGE CARDIOVASCULAR MICROFLUIDIC ORGAN-CHIP

(71) Applicant: CEDARS-SINAI MEDICAL CENTER, Los Angeles, CA (US)

(72) Inventors: Arun Sharma, Los Angeles, CA (US); Samuel Sances, Santa Monica, CA (US); Clive Svendsen, Pacific Palisades, CA (US)

(73) Assignee: CEDARS-SINAI MEDICAL CENTER, Los Angeles, CA (US)

(21) Appl. No.: 17/921,217

(22) PCT Filed: Apr. 30, 2021
(86) PCT No.: PCT/US2021/030128

§ 371 Date: Oct. 25, 2022

Related U.S. Application Data
(60) Provisional application No. 63/019,058, filed on May 1, 2020.

(51) Int. Cl. C12N 5/077; C12M 3/06; C12M 3/00; C12M 1/00; C12N 5/071; G01N 33/50

(52) U.S. Cl. CPC C12N 5/0657; C12M 21/08; C12M 23/16; C12M 29/04; C12N 5/069; C12N 5/0697; G01N 33/5014; C12N 2506/45

(57) ABSTRACT

Described herein is a human, cardiovascular platform for assessing cardiotoxicity of novel/existing chemotherapeutic agents that takes advantage of microfluidic organ chip systems to examine interaction between hiPSC-derived cardiovascular cells in an integrated system. Human induced pluripotent stem cell derived cardiomyocytes (hiPSC-CMs) and human induced pluripotent stem cell derived endothelial cells (hiPSC-ECs) can serve as an in-vitro platform for assessing disease pathology, including infectious disease, evaluate drug efficacy, toxicity, cardiotoxicity and cardioprotection. This includes evaluating VEGFR2/PDGFR-inhibiting tyrosine kinase inhibitors and drug efficacy in a viral infection model, including coronaviruses. They are scalable, functionally-active cell types that mimic the cells comprising the myocardium and systemic vasculature.

### CROSS-REFERENCE TO RELATED APPLICATIONS

This application includes a claim of priority under 35 U.S.C. §119(e) to U.S. provisional patent application No. 63/019,058, filed May 1, 2020, the entirety of which is hereby incorporated by reference.

### STATEMENT REGARDING FEDERALLY SPONSORED RESEARCH OR DEVELOPMENT

This invention was made with government support under Grant No. NS105703 awarded by National Institutes of Health. The government has certain rights in the invention.

### FIELD OF INVENTION

This invention relates to devices and methods of using the device for drug screening and for modeling the cardiac diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Traditional methods for preclinical evaluation of drug efficacy and toxicity, including cardiotoxicity, use animal models, which are expensive, low-throughput, and exhibit species-specific differences in cardiovascular physiology. Alternative models utilize non-cardiovascular cells expressing cardiovascular genes. One issue with non-cardiac cells is that they lack cardiomyocyte-specific components such as sarcomeres and ion channel subunits. Primary human cardiomyocytes (CMs) could be used for preclinical evaluation of drug cardiotoxicity, but their access is limited and their long-term maintenance is a technical challenge. Also, since adult human CMs are mitotically inactive, they cannot be expanded in vitro to obtain enough cells for drug screening. The most predictive models for drug cardiotoxicity need to reproduce the complex spatial distribution of the CMs, endothelial cells (ECs), and support cells of the adult human heart. However, primary human heart preps are too costly, too difficult to maintain, and too low-throughput to be implemented early during drug development. Thus, there remains significant interest in developing alternative human cardiovascular cell types for preclinical drug testing that are cost-effective, predictive, and can hence be implemented early in the drug discovery process.

Described herein is a multi-lineage, fully-integrated cardiovascular organ-chip comprised of both human induced pluripotent stem cell-derived heart muscle cells, or cardiomyocytes (hiPSC-CMs) and endothelial blood vessel cells (hiPSC-ECs) that serves as an improved predictive platform to determine drug efficacy, toxicity, cardiotoxicity and cardioprotection over mono-lineage cell culture. This work provides a fully-integrated, multi-lineage, human organ-chip for modeling disease pathology, including infection disease, examining drug toxicity and efficacy, and aids the development of new therapeutics and assist clinicians in choosing treatment regimens.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a device, comprising: a membrane comprising a top surface and a bottom surface; a first channel in fluidic communication with the top surface of the membrane; a second channel in fluidic communication with the bottom surface of the membrane, wherein the first and second channels each comprises a surface that is parallel to the membrane; induced pluripotent stem cell derived-endothelial cells (iPSC-ECs) in the first channel or the second channel; induced pluripotent stem cell derived-cardiac cells (iPSC-CCs) in the first channel or the second channel, wherein the iPSC-ECs and the iPSC-CCs are in different channels.

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs).

In various embodiments, the device can further comprise at least one inlet port adapted for fluid entering the at least one inlet port; and at least one outlet port adapted for fluid exiting the at least one outlet port.

Various embodiments of the present invention provide for a device, comprising: a top chamber; a bottom chamber; a membrane between the top chamber and the bottom chamber; a first channel fluidically coupled to the top chamber; a second channel fluidically coupled to the bottom chamber; induced pluripotent stem cell derived-endothelial cells (iPSC-vECs) in the first channel or the second channel; and induced pluripotent stem cell derived-cardiac cells (iPSC-CCs) in the first channel or the second channel, wherein the iPSC-ECs and the iPSC-CCs are in different channels.

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs).

In various embodiments, the first and second channels can comprise polydimethylciloxane.

In various embodiments, the iPSC-CCs can be in the first channel and the iPSC-ECs can be in the second channel. In various embodiments, the iPSC-CMs can be in the first channel and the iPSC-vECs can be in the second channel.

In various embodiments, the first channel and the second channel can be microfluidic channels.

Various embodiments of the present invention provide for an organ chip device, comprising: induced pluripotent stem cell derived-endothelial cells (iPSC-ECs); induced pluripotent stem cell derived-cardiac cells (iPSC-CCs); and a membrane separating the iPSC-ECs and iPSC-CCs.

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs).

In various embodiments, the membrane can comprise polydimethylciloxane.

In various embodiments, the device can further comprise one or more gels and the iPSC-ECs or the iPSC-CCs, or both separately having been seeded on top of or into the one or more gels.

In various embodiments, the iPSC-ECs and iPSC-CCs can be patient specific.

In various embodiments, the iPSC-CCs can express a fluorescent reporter.

In various embodiments, the iPSC-ECs can be alpha-actinin-GFP iPSCs, or the iPSC-CCs can be alpha-actinin-GFP iPSC-CCs, or both.

In various embodiments, the iPSC-ECs can be human iPSC-ECs (hiPSC-ECs). In various embodiments, the iPSC-CCs can be human iPSC-CCs (hiPSC-CCs).

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs).

Various embodiments of the present invention provide for a method of assessing a test agent, comprising: contacting the test agent to a device the present invention; measuring a parameter; and assessing the test agent based on the measured parameter.

In various embodiments, the test agent can be a chemotherapeutic agent. In various embodiments, the test agent can be a tyrosine kinase inhibitor (TKI). In various embodiments, the test agent can be a VEGFR2/PDGFR-inhibiting tyrosine kinase inhibitor. In various embodiments, the test agent can be an infectious agent.

In various embodiments, assessing the test agent can comprise assessing cardiotoxicity of the test agent.

In various embodiments, measuring the parameter can comprise measuring a phenotype of interest, expression level of a gene of interest, or expression level of a protein of interest, or combinations thereof.

In various embodiments, measuring the parameter can comprise measuring fluorescence. In various embodiments, measuring the parameter can comprise measuring sarcomere contractility or cell contractility. In various embodiments, measuring the parameter can comprise measuring TEER resistance. In various embodiments, measuring the parameter can comprise measuring expression levels of MYH7, MYH6, or both. In various embodiments, measuring the parameter can comprise measuring expression levels of CDH5(CD144), PECAM1 (DC31), KDR (VEGFR2), NR2F2 (COUP-TFII), EFNB2, TEK (TIE2), MYH6, MYH7, ACTN2, or TNNT2 or combinations thereof. In various embodiments, measuring the parameter can comprise measuring cell viability. In various embodiments, measuring the parameter can comprise obtaining calcium imaging of the iPSC-ECs or the iPSC-CCs, or both.

In various embodiments, contacting the iPSC-CCs, iPSC-ECs, or both, with the test agent can comprise culturing the iPSC-CCs, iPSC-ECs, or both, in the presence of liver-conditioned media.

In various embodiments, contacting the iPSC-CCs, iPSC-ECs, or both, with the test agent can comprise further culturing the iPSC-CCs, iPSC-ECs, or both with an agent capable of activating Akt signaling. In various embodiments, contacting the iPSC-CCs, iPSC-ECs, or both, with the test agent can comprise culturing the iPSC-CCs, iPSC-ECs, or both in the presence of culture media flowing through the device.

In various embodiments, iPSC-ECs and iPSC-CCs can be patient specific and the method models patient-specific parameters.

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs).

Various embodiments of the present invention provide for a method of producing a device the present invention, comprising: seeding induced pluripotent stem cell derived cardiac cells (iPSC-CCs) on one surface of the membrane in the device; and seeding induced pluripotent stem cell derived endothelial cells (iPSC-ECs) on the other surface of the membrane in the device; OR seeding induced pluripotent stem cell derived cardiac cells (iPSC-CCs) in one chamber in the device; and seeding induced pluripotent stem cell derived endothelial cells (iPSC-ECs) in the other chamber in the device.

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the iPSC-ECs can be induced pluripotent stem cell derived-vascular endothelial cells (iPSC-vECs); and the iPSC-CCs can be induced pluripotent stem cell derived-cardiomyocytes (iPSC-CMs).

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts multi-lineage, cardiovascular, microfluidic organ-chips in accordance with various embodiments of the present invention.

FIG. 2 shows hiPSC-CMs expressing standard cardiac markers in accordance with various embodiments of the present invention.

FIG. 3 shows hiPSC-CMs expressing standard cardiac markers in accordance with various embodiments of the present invention.

FIG. 4 shows CD144+ hiPSC-derived Vascular Endothelial Cell (EC) Differentiation.

FIG. 5 shows banked CD144+ hiPSC-ECs express standard vascular EC markers in accordance with various embodiments of the present invention.

FIG. 6 shows transendothelial electrical resistance (TEER) of CD144+ hiPSC-ECs in accordance with various embodiments of the present invention.

FIG. 7 shows seeding of hiPSC-CMs (top channel) and hiPSC-ECs (bottom channel) in accordance with various embodiments of the present invention.

FIG. 8 shows immunofluorescence of preliminary organ chip in accordance with various embodiments of the present invention.

FIG. 9 shows immunofluorescence of hiPSC-CMs/ECs that survive >50 days on organ chip in accordance with various embodiments of the present invention.

FIG. 10 shows CV Chip + media flow to mimic circulation and enhance maturation in accordance with various embodiments of the present invention.

FIG. 11 shows the incorporation of ACTN2-GFP hiPSC-CM reporter line (Coriell) to assess CM maturity on CV Chip in accordance with various embodiments of the present invention. Right panel from Sharma et al. 2018, Curr Protoc Hum Gen.

FIG. 12 shows ACTN2-GFP hiPSC-CMs mark sarcomeres in accordance with various embodiments of the present invention.

FIG. 13 shows ACTN2-GFP hiPSC-CMs mark sarcomeres in accordance with various embodiments of the present invention.

FIG. 14 shows ACTN2-GFP hiPSC-CMs mark sarcomeres in accordance with various embodiments of the present invention.

FIG. 15 shows that calcium Imaging can be Performed on CV-Chip CMs in accordance with various embodiments of the present invention.

FIG. 16 shows that Barrier Permeability is Orders of Magnitude Higher in CV-Chips than BBB-Chips in accordance with various embodiments of the present invention. Left panels from Vatine et al. 2019, Cell Stem Cell for comparison.

FIGS. 17A and 17B show flow conditions enhances hiPSC-CM contractility on CV-Chips in accordance with various embodiments of the present invention.

FIG. 18 shows PCA of Static vs. Flow CV Chip RNAseq (CMs, vECs) in accordance with various embodiments of the present invention.

FIG. 19 shows hiPSC-CMs and vECs upregulate markers of maturity under flow in accordance with various embodiments of the present invention.

FIG. 20A shows CV-Chip with 30 µL/hr media flow for 10 days in accordance with various embodiments of the present invention.

FIG. 20B shows hiPSC-vECs aggregate to form “capillary-like” structures under active flow in accordance with various embodiments of the present invention.

FIG. 21 shows hiPSC-vECs aggregate to form “capillary-like” structures under active flow in accordance with various embodiments of the present invention.

FIG. 22 shows longitudinal Analysis Shows Long-Term Stability of CV-Chips: No increased cytotoxicity, and enhanced metabolic output in accordance with various embodiments of the present invention.

FIG. 23 depicts hiPSC-CM Longitudinal Beat Rate Assessment in accordance with various embodiments of the present invention.

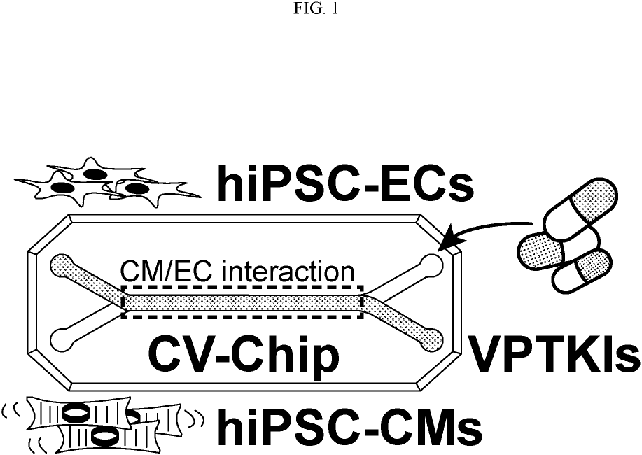

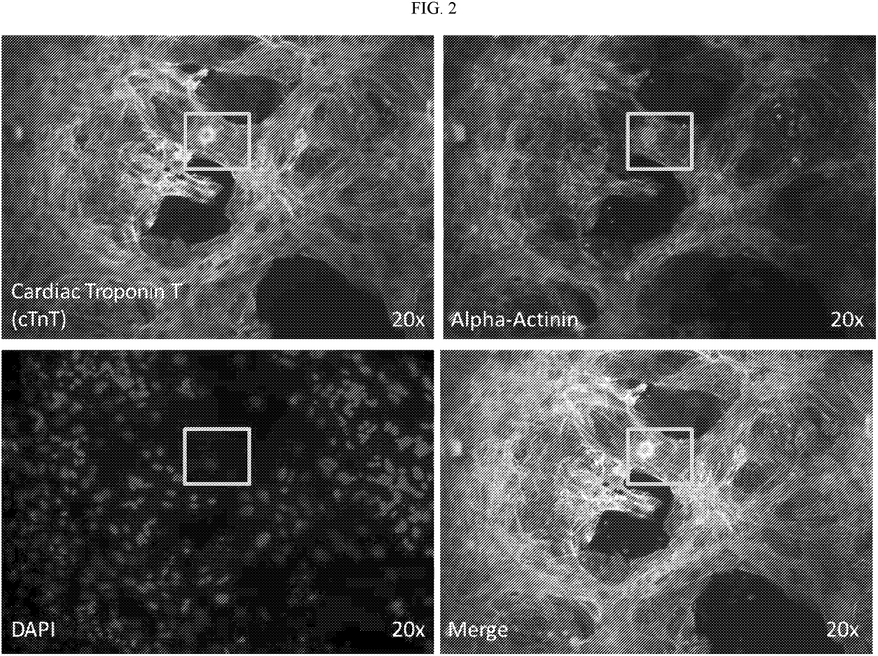

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

As used herein the term “organ chip” (also referred to as “organ on chip”) refers to a microfluidic culture device are capable of recapitulating the microarchitecture and functions of living organs.

New pre-clinical methods have enabled cardiotoxic compounds to be identified early in drug development. Human induced pluripotent stem cell (hiPSC) derived cardiomyocytes (CMs) and endothelial cells (ECs) can screen for drug-induced alterations in CV cell function and survival. However, existing hiPSC models for CV drug toxicity utilize isolated hiPSC-CMs or hiPSC-vascular ECs, without mimicking a circulatory system connecting the cell types or pre-metabolizing the drugs like what occurs in vivo. There is a great need in the art for better in vitro models of drug toxicity would incorporate hiPSC-derived CMs and ECs in an integrated system whereby metabolically-activated drugs can flow from hiPSC-ECs, to represent systemic vasculature, to hiPSC-CMs, representing heart muscle (myocardium). This would be useful for testing cardiotoxicities of drugs such as VEGFR2/PDGFR-inhibiting tyrosine kinase inhibitors (VPTKls), small molecule drugs used for treating various types of cancers, but which exhibit dual EC/CM toxicities.

Traditional methods for preclinical evaluation of drug cardiotoxicity use animal models, which are expensive, low-throughput, and exhibit species-specific differences in cardiovascular physiology. Alternative models utilize non-cardiovascular cells expressing cardiovascular genes. One issue with non-cardiac cells is that they lack cardiomyocyte-specific components such as sarcomeres and ion channel subunits. Primary human cardiomyocytes (CMs) could be used for preclinical evaluation of drug cardiotoxicity, but their access is limited and their long-term maintenance is a technical challenge. Also, since adult human CMs are mitotically inactive, they cannot be expanded in vitro to obtain enough cells for drug screening. The most predictive models for drug cardiotoxicity need to reproduce the complex spatial distribution of the CMs, endothelial cells (ECs), and support cells of the adult human heart. However, primary human heart preps are too costly, too difficult to maintain, and too low-throughput to be implemented early during drug development. Thus, there remains significant interest in developing alternative human cardiovascular cell types for preclinical drug testing that are cost-effective, predictive, and can hence be implemented early in the drug discovery process.

Further described herein is a specific context where the aforementioned innovation can support discovery of toxicity. In particular, VEGFR2/PDGFR- inhibiting tyrosine kinase inhibitor (VPTKI)-induced cardiotoxicity. Vascular endothelial growth factor receptor (VEGFR2) and platelet- derived growth factor receptor (PDGFR) are two major angiogenic cell signaling proteins that are hyper-activated in cancer. Chemotherapeutic agents such as dual VEGFR2/PDGFR-inhibiting tyrosine kinase inhibitors (VPTKIs) have increased the 5-year survival rates for patients with cancers such as leukemia and renal cell carcinoma to greater than 68%. However, VPTKI utility is limited by cardiovascular toxicity. The cardiovascular toxicity of chemotherapeutic drugs is relevant for cancer patients whose life expectancy is restored to normal but who may be left with cardiovascular abnormalities that can present years later. Cardiovascular toxicity can include induced arrhythmias, cardiomyocyte (CM) apoptosis, heart failure, endothelial cell (EC) dysfunction, acute arterial or venous thrombotic events, bleeding, or downstream hypertension. Patients with chemotherapeutic cardiotoxicity often have few options for treatment because they have a combination of vascular toxicities and dilated or restrictive cardiomyopathies. At present, it is impossible to predict which patients will be affected or protect patients who could suffer from VPTKI-induced cardiovascular toxicity (VPTKI-TOX).

Mechanisms of VPTKI-induced cardiovascular toxicity (VPTKI-TOX) are poorly understood. Despite more than a decade of research, the mechanisms of VPTKI-TOX are not well-defined. Tyrosine kinases are ubiquitous intracellular proteins that play critical roles in cell signaling pathways involved in metabolism, injury response, growth, and differentiation. Tyrosine kinases add a phosphate group to a protein (phosphorylation) on a tyrosine, “activating” the protein. Tyrosine kinase receptor antagonists act by inhibition of tyrosine kinase pathways. These pathways include the angiogenic VEGFR and PDGFR pathways that are overactive or unregulated in cancerous cells, and VPTKIs target these cell signaling pathways. Older VPTKIs tend to target multiple signaling pathways, while the newer generation of drugs is more selective and targets VEGFR signaling alone, in the hope of eliciting a more potent anti- angiogenic effect.

TABLE 1

Clinical doses of some VEGFR2/PDGFR-inhibiting tyrosine kinase inhibitors (VPTKIs) and relevant cell culture doses

| Drug | MW | Peak plasma concentration (Cmax) | Molar Cmax | Common dose | Cell culture dose range |
|---|---|---|---|---|---|
| sunitinib | 398.474 | 81.9 ng/mL | 1.2 µM | 50 mg once daily | 0.1-5 uM |
| sorafenib | 464.825 | 1.8 µg/mL | 2.99 µM | 400 mg twice daily | 1-10 uM |
| pazopanib | 437.517 | 1.96 µg/mL | 3.65 µM | 800 mg once daily | 1-10 uM |
| vandetanib | 475.36 | 960 ng/mL | 4.26 µM | 300 mg once daily | 1-10 uM |
| axitinib | 386.469 | 12.6 ng/mL | 0.9 µM | 5 mg twice daily | 0.1-5 uM |
| ponatinib | 532.56 | 73 ng/mL | 0.14 µM | 45 mg once daily | 0.1-5 uM |
| regorafenib | 500.83 | 2.5 µg/mL | 8.08 µM | 160 mg once daily | 1-10 uM |
| lenvatinib | 426.85 | 660 ng/mL | 1.54 µM | 24 mg once daily | 1-10 uM |

Currently, there are more than 20 VPTKIs in development, with cancer being the major clinical indication. The first VPTKI approved was sorafenib, followed by sunitinib, pazopanib, vandetanib, axitinib, ponatinib, regorafenib, and lenvatinib (See Table 1). Other VPTKIs, such as cediranib and tivozanib are in development. VPTKIs including sorafenib, sunitinib, and pazopanib may cause heart failure or vascular toxicity. Hypertension is also a highly prevalent cardiotoxicity associated with VPTKIs. The VPTKI ponatinib has an FDA black box warning for arterial occlusions and venous thromboembolisms as well as hepatotoxicity, and it was temporarily withdrawn from use. With the pharmaceutical industry’s aggressive pursuit of VPTKIs, effective methods for preclinically assessing VPTKI-TOX could greatly reduce development costs.

There is a growing need for cardioprotective agents against VPTKI-TOX and cardiotoxicity elicited by other chemotherapeutic agents. The pursuit of cardioprotective treatments against chemotherapy-induced cardiotoxicity has dramatically increased since the initial usage of anthracyclines (Table 2). Thus far, only a single cardioprotectant is approved for clinical use (dexrazoxane). Others have been tested clinically, and many more remain in preclinical development. The majority of chemotherapeutic cardioprotectants have been tested in the setting of anthracycline administration, given the common usage and long-term existence of doxorubicin and daunorubicin in cancer treatment regimens. Cardioprotectants can be administered either alone or in combination with chemotherapeutic agents. However, there remains no specific cardioprotective agent that can counteract the cardiotoxic and vasculotoxic effects of VPTKIs. Additionally, most cardioprotective agents treat the resulting symptoms of cardiotoxicity, as opposed to specifically treating the underlying mechanisms driving VPTKI-TOX. Thus, there is significant interest in creating VPTKI-specific cardioprotective agents.

TABLE 2

Cardioprotective strategies against chemotherapy-induced cardiotoxicity

| | | |
|---|---|---|
| Drug | Protective against | Mechanism |
| dexrazoxane | Anthracycline cardiotoxicity | Iron chelation, reduction of superoxide radicals |
| N-acetylcystine | Anthracycline cardiotoxicity | Scavenging free radicals and reactive oxygen species |
| ACE inhibitors | Hypertension, heart failure | Inhibition of angiotensin-converting enzyme |
| Angiotensin receptor blockers | Hypertension, heart failure | Block angiotensin II AT1 receptors |
| Spironolactone | Anthracycline cardiotoxicity | Blocking the renin-angiotensin-aldosterone system |
| Beta-blockers | Anthracycline cardiotoxicity | Anti-oxidant and anti-inflammation properties |
| Atorvastatin | Anthracycline cardiotoxicity | Anti-oxidative properties |

Accurate pre-clinical screening platforms for identifying cardiotoxic VPTKIs are needed, and hiPSC-derived cardiovascular cells have emerged as a potential solution. Traditional methods for preclinical evaluation of drug cardiotoxicity use animal models, which are expensive, low-throughput, and exhibit species-specific differences in cardiovascular physiology. Alternative models utilize non- cardiovascular cells expressing cardiovascular genes. One issue with non-cardiac cells is that they lack cardiomyocyte-specific components such as sarcomeres and ion channel subunits. Primary human cardiomyocytes (CMs) could be used for preclinical evaluation of drug cardiotoxicity, but their access is limited and their long-term maintenance is a technical challenge. Also, since adult human CMs are mitotically inactive, they cannot be expanded in vitro to obtain enough cells for drug screening. The most predictive models for drug cardiotoxicity need to reproduce the complex spatial distribution of the CMs, endothelial cells (ECs), and support cells of the adult human heart. However, primary human heart preps are too costly, too difficult to maintain, and too low-throughput to be implemented early during drug development. Thus, there remains significant interest in developing alternative human cardiovascular cell types for preclinical drug testing that are cost-effective, predictive, and can hence be implemented early in the drug discovery process.

Described herein is a combination of iPSC-CMs and iPSC-ECs (particularly, hiPSC-CMs and hiPSC-vascular ECs (hiPSC-vECs)) which can serve as an in-vitro platform for assessing disease pathology, including infectious disease, evaluate drug efficacy, toxicity, cardiotoxicity and cardioprotection. Also described herein are models for assessing VPTKI-TOX and drug efficacy. They are scalable, functionally-active cell types that mimic the cells comprising the myocardium and systemic vasculature. The ability to accurately identify drugs in a safe, preclinical setting will potentially prevent vulnerable cancer patients from developing dangerous cardiovascular side effects due to chemotherapy.

### Devices

Various embodiments of the present invention provide for a device, comprising: a membrane comprising a top surface and a bottom surface; a first channel in fluidic communication with the top surface of the membrane; a second channel in fluidic communication with the bottom surface of the membrane, wherein the first and second channels each comprises a surface that is parallel to the membrane; induced pluripotent stem cell derived-endothelial cells (iPSC-ECs) in the first channel or the second channel; induced pluripotent stem cell derived-cardiac cells (iPSC-CCs) in the first channel or the second channel, wherein the iPSC-ECs and the iPSC-CCs are in different channels.

In various embodiments, the iPSC-ECs are induced pluripotent stem cell derived-vascular endothelial cells (iPSC-vECs); and the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the device further comprises at least one inlet port adapted for fluid entering the at least one inlet port; and at least one outlet port adapted for fluid exiting the at least one outlet port.

In various embodiments, the first and second channels comprise polydimethylciloxane. In various embodiments, the membrane comprises polydimethylciloxane.

In various embodiments, the iPSC-CCs are in the first channel and the iPSC-ECs are in the second channel.

In various embodiments, the first channel and the second channel are microfluidic channels.

In various embodiments, the device further comprises one or more gels and the iPSC-ECs or the iPSC-CCs, or both separately having been seeded on top of or into the one or more gels.

In various embodiments, the iPSC-ECs and iPSC-CCs are patient specific.

In various embodiments, the iPSC-CCs express a fluorescent reporter. In various embodiments, the iPSC-ECs are alpha-actinin-GFP iPSCs, or the iPSC-CCs are alpha-actinin-GFP iPSC-CCs, or both.

In various embodiments, the iPSC-ECs are human iPSC-ECs (hiPSC-ECs). In various embodiments, the iPSC-CCs are human iPSC-CCs (hiPSC-CCs)

Various embodiments of the present invention provide for a device, comprising: a membrane comprising a top surface and a bottom surface; a first channel in fluidic communication with the top surface of the membrane; a second channel in fluidic communication with the bottom surface of the membrane, wherein the first and second channels each comprise a surface that is parallel to the membrane; induced pluripotent stem cell derived-vascular endothelial cells (iPSC-vECs) in the first channel or the second channel; and induced pluripotent stem cell derived-cardiomyocytes (iPSC-CMs) in the first channel or the second channel, wherein the iPSC-vECs and the iPSC-CMs are in different channels.

In various embodiments, the device further comprises: at least one inlet port adapted for fluid entering the at least one inlet port; and at least one outlet port adapted for fluid exiting the at least one outlet port.

In various embodiments, the first and second channels comprise polydimethylciloxane. In various embodiments, the membrane comprises polydimethylciloxane.

In various embodiments, the iPSC-CMs are in the first channel and the iPSC-vECs are in the second channel. In various embodiments, the first channel and the second channel are microfluidic channels.

In various embodiments, the device further comprises one or more gels and the iPSC-vECs or the iPSC-CMs, or both separately having been seeded on top of or into the one or more gels.

In various embodiments, the iPSC-vECs and iPSC-CMs are patient specific.

In various embodiments, the iPSC-CMs express a fluorescent reporter. In various embodiments, the iPSC-vECs are alpha-actinin-GFP iPSC-vECs. In various embodiments, the iPSC-CMs are alpha-actinin-GFP iPSC-CMs. In various embodiments, both the iPSC-CMs are alpha-actinin-GFP iPSC-CMs and the iPSC-vECs are alpha-actinin-GFP iPSC-vECs.

In various embodiments, the iPSC-vECs are human iPSC-vECs (hiPSC-vECs). In various embodiments, the iPSC-CMs are human iPSC-CMs (hiPSC-vECs).

In various embodiments, the device comprises: a top surface and a bottom surface; a first microfluidic channel in fluidic communication with the top surface of the membrane; a second microfluidic channel in fluidic communication with the bottom surface of the membrane, wherein the first and second microfluidic channels each comprises a surface that is parallel to the membrane; at least one inlet port adapted for fluid entering the at least one inlet port; and at least one outlet port adapted for fluid exiting the at least one outlet port; human induced pluripotent stem cell derived-cardiomyocytes (hiPSC-CMs) in the first microfluidic channel; human induced pluripotent stem cell derived-vascular endothelial cells (hiPSC-vECs) in the second microfluidic channel, wherein the hiPSC-CMs are seeded on top of a gel or in a gel and the hiPSC-vECs are seeded on top of a gel or in a gel.

Various embodiments of the present invention provide for a device, comprising: a top chamber; a bottom chamber; a membrane between the top chamber and the bottom chamber; a first channel fluidically coupled to the top chamber; a second channel fluidically coupled to the bottom chamber; induced pluripotent stem cell derived-endothelial cells (iPSC-vECs) in the first channel or the second channel; and induced pluripotent stem cell derived-cardiac cells (iPSC-CCs) in the first channel or the second channel, wherein the iPSC-ECs and the iPSC-CCs are in different channels.

In various embodiments, the iPSC-ECs are induced pluripotent stem cell derived-vascular endothelial cells (iPSC-vECs); and the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the first and second channels comprise polydimethylciloxane. In various embodiments, the membrane comprises polydimethylciloxane.

In various embodiments, the iPSC-CCs are in the first channel and the iPSC-ECs are in the second channel. In various embodiments, the iPSC-CMs are in the first channel and the iPSC-vECs are in the second channel.

In various embodiments, the first channel and the second channel are microfluidic channels.

In various embodiments, the device further comprises one or more gels and the iPSC-ECs or the iPSC-CCs, or both separately having been seeded on top of or into the one or more gels.

In various embodiments, the iPSC-ECs and iPSC-CCs are patient specific.

In various embodiments, the iPSC-CCs express a fluorescent reporter. In various embodiments, the iPSC-ECs are alpha-actinin-GFP iPSCs, or the iPSC-CCs are alpha-actinin-GFP iPSC-CCs, or both.

In various embodiments, the iPSC-ECs are human iPSC-ECs (hiPSC-ECs). In various embodiments, the iPSC-CCs are human iPSC-CCs (hiPSC-CCs)

Various embodiments of the present invention provide for a device, comprising: a top chamber; a bottom chamber; a membrane between the top chamber and the bottom chamber; a first channel fluidically coupled to the top chamber; and a second channel fluidically coupled to the bottom chamber induced pluripotent stem cell derived-vascular endothelial cells (iPSC-vECs) in the first channel or the second channel; and induced pluripotent stem cell derived-cardiomyocytes (iPSC-CMs) in the first channel or the second channel, wherein the iPSC-vECs and the iPSC-CMs are in different channels.

In various embodiments, the device further comprises: at least one inlet port adapted for fluid entering the at least one inlet port; and at least one outlet port adapted for fluid exiting the at least one outlet port.

In various embodiments, the first and second channels comprise polydimethylciloxane. In various embodiments, the membrane comprises polydimethylciloxane.

In various embodiments, the iPSC-CMs are in the first channel and the iPSC-vECs are in the second channel. In various embodiments, the first channel and the second channel are microfluidic channels.

In various embodiments, the device further comprises one or more gels and the iPSC-vECs or the iPSC-CMs, or both separately having been seeded on top of or into the one or more gels.

In various embodiments, the iPSC-vECs and iPSC-CMs are patient specific.

In various embodiments, the iPSC-CMs express a fluorescent reporter. In various embodiments, the iPSC-vECs are alpha-actinin-GFP iPSC-vECs. In various embodiments, the iPSC-CMs are alpha-actinin-GFP iPSC-CMs. In various embodiments, both the iPSC-CMs are alpha-actinin-GFP iPSC-CMs and the iPSC-vECs are alpha-actinin-GFP iPSC-vECs.

In various embodiments, the iPSC-vECs are human iPSC-vECs (hiPSC-vECs). In various embodiments, the iPSC-CMs are human iPSC-CMs (hiPSC-vECs).

In various embodiments, the device comprises a top chamber; a bottom chamber; a membrane between the top chamber and the bottom chamber; a first channel fluidically coupled to the top chamber; and a second channel fluidically coupled to the bottom chamber; at least one inlet port adapted for fluid entering the at least one inlet port; and at least one outlet port adapted for fluid exiting the at least one outlet port; human induced pluripotent stem cell derived-cardiomyocytes (hiPSC-CMs) seeded on top of a gel or in a gel in the first channel; and human induced pluripotent stem cell derived-vascular endothelial cells (hiPSC-vECs) seeded on top of a gel or in a gel in the second channel.

Various embodiments provide for an organ chip device, comprising: induced pluripotent stem cell derived-endothelial cells (iPSC-ECs); induced pluripotent stem cell derived-cardiac cells (iPSC-CCs); and a membrane separating the iPSC-ECs and iPSC-CCs.

In various embodiments, the device further comprises a first channel and a second channel. In various embodiments, the device further comprises: at least one inlet port adapted for fluid entering the at least one inlet port; and at least one outlet port adapted for fluid exiting the at least one outlet port.

In various embodiments, the iPSC-ECs are induced pluripotent stem cell derived-vascular endothelial cells (iPSC-vECs); and the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

In various embodiments, the membrane comprises polydimethylciloxane. In various embodiments, the membrane comprises polydimethylciloxane.

In various embodiments, the device further comprises one or more gels and the iPSC-ECs or the iPSC-CCs, or both separately having been seeded on top of or into the one or more gels.

In various embodiments, the iPSC-CCs are in the first channel and the iPSC-ECs are in the second channel. In various embodiments, the first channel and the second channel are microfluidic channels.

In various embodiments, the iPSC-ECs and iPSC-CCs are patient specific.

In various embodiments, the iPSC-CCs express a fluorescent reporter. In various embodiments, the iPSC-ECs are alpha-actinin-GFP iPSCs, or the iPSC-CCs are alpha-actinin-GFP iPSC-CCs, or both.

In various embodiments, the iPSC-ECs are human iPSC-ECs (hiPSC-ECs). In various embodiments, the iPSC-CCs are human iPSC-CCs (hiPSC-CCs)

Various embodiments provide for an organ chip device, comprising: induced pluripotent stem cell derived-vascular endothelial cells (iPSC-vECs); induced pluripotent stem cell derived-cardiomyocytes (iPSC-CMs); and a membrane separating the iPSC-vECs and iPSC-CMs.

In various embodiments, the device further comprises a first channel and a second channel. In various embodiments, the device further comprises: at least one inlet port adapted for fluid entering the at least one inlet port; and at least one outlet port adapted for fluid exiting the at least one outlet port.

In various embodiments, the first and second channels comprise polydimethylciloxane. In various embodiments, the membrane comprises polydimethylciloxane.

In various embodiments, the iPSC-CMs are in the first channel and the iPSC-vECs are in the second channel. In various embodiments, the first channel and the second channel are microfluidic channels.

In various embodiments, the device further comprises one or more gels and the iPSC-vECs or the iPSC-CMs, or both separately having been seeded on top of or into the one or more gels.

In various embodiments, the iPSC-vECs and iPSC-CMs are patient specific.

In various embodiments, the iPSC-CMs express a fluorescent reporter. In various embodiments, the iPSC-vECs are alpha-actinin-GFP iPSC-vECs. In various embodiments, the iPSC-CMs are alpha-actinin-GFP iPSC-CMs. In various embodiments, both the iPSC-CMs are alpha-actinin-GFP iPSC-CMs and the iPSC-vECs are alpha-actinin-GFP iPSC-vECs.

In various embodiments, the iPSC-vECs are human iPSC-vECs (hiPSC-vECs). In various embodiments, the iPSC-CMs are human iPSC-CMs (hiPSC-vECs).

### Methods of Accessing a Test Agent

Various embodiments of the present invention provide for a method of assessing a test agent, comprising: contacting the test agent to any one of the devices of the present invention as described herein; measuring a parameter; and assessing the test agent based on the measured parameter. The devices of the present invention as described herein comprises induced pluripotent stem cell derived-endothelial cells (iPSC-ECs) and induced pluripotent stem cell derived-cardiac cells (iPSC-CCs) as discussed herein. In various embodiments, the iPSC-ECs are induced pluripotent stem cell derived-vascular endothelial cells (iPSC-vECs); and the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs). In various embodiments, the the iPSC-ECs are induced pluripotent stem cell derived-vascular endothelial cells (iPSC-vECs); and the iPSC-CCs are induced pluripotent stem cell derived-cardiomyocytes (iPSC-CMs).

Assessing the agent can include assessing the effects the agent may have on the heart or cardiac cells. In certain instances, the test agent may be identified as a beneficial agent. In other instances, the test agent may be identified as cardiotoxic. In still other instances, the test agent may be identified as cardioprotective.

In various embodiments, the test agent is a chemotherapeutic agent. Examples of chemotherapeutic agents include anthracyclines, cytotoxic agents (e.g., 5-fluorouracil, cisplatin, carboplatin, methotrexate, daunorubicin, doxorubicin (Adriamycin®), vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic akylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

In various embodiments, the test agent is a tyrosine kinase inhibitor (TKI). In various embodiments, the test agent is a VEGFR2/PDGFR-inhibiting tyrosine kinase inhibitor. Examples of TKIs include but are not limited to Avapritinib, Capmatinib, Pemigatinib, Ripretinib, Selpercatinib, Selumetinib, Tucatinib, Entrectinib, Erdafitinib, Fedratinib, Pexidartinib, Upadacitinib, Zanubrutinib, Baricitinib, Binimetinib, Dacomitinib, Fostamatinib, Gilteritinib, Larotrectinib, Lorlatinib, Acalabrutinib, Brigatinib, Midostaurin, Neratinib, Alectinib, Cobimetinib, Lenvatinib, Osimertinib, Ceritinib, Nintedanib, Afatinib, Ibrutinib, Trametinib, Axitinib, Bosutinib, Cabozantinib, Ponatinib, Regorafenib, Tofacitinib, Crizotinib, Ruxolitinib, Vandetanib, Pazopanib, Lapatinib, Nilotinib, Dasatinib, Sunitinib, Sorafenib, Erlotinib, Gefitinib, and Imatinib.

In various embodiments, the test agent is an infectious agent. Examples of infectious agents include viruses, bacteria, fungi, and parasites.

In various embodiments, assessing the test agent comprises assessing cardiotoxicity of the test agent.

In various embodiments, measuring the parameter comprises measuring a phenotype of interest, expression level of a gene of interest, or expression level of a protein of interest, or combinations thereof.

In various embodiments, measuring the parameter comprises measuring fluorescence. In various embodiments, measuring the parameter comprises measuring sarcomere contractility. In various embodiments, measuring the parameter comprises measuring cell contractility. In various embodiments, measuring the parameter comprises measuring TEER resistance.

In various embodiments, measuring the parameter comprises measuring expression levels of MYH7, MYH6, or both. In various embodiments, measuring the parameter comprises measuring expression levels of CDH5(CD144), PECAM1 (DC31), KDR (VEGFR2), NR2F2 (COUP-TFII), EFNB2, TEK (TIE2), MYH6, MYH7, ACTN2, or TNNT2 or combinations thereof.

In various embodiments, measuring the parameter comprises measuring cell viability. In various embodiments, measuring the parameter comprises obtaining calcium imaging of the iPSC-ECs or the iPSC-CCs, or both.

In various embodiments, contacting the iPSC-CCs, iPSC-ECs, or both, with the test agent comprises culturing the iPSC-CCs, iPSC-ECs, or both, in the presence of liver-conditioned media. In various embodiments, contacting the iPSC-CCs, iPSC-ECs, or both, with the test agent comprises further culturing the iPSC-CCs, iPSC-ECs, or both with an agent capable of activating Akt signaling. In various embodiments, contacting the iPSC-CCs, iPSC-ECs, or both, with the test agent comprises culturing the iPSC-CCs, iPSC-ECs, or both in the presence of culture media flowing through the device.

In various embodiments, iPSC-ECs and iPSC-CCs are patient specific and the method models patient-specific parameters.

In various embodiments, measuring the parameter comprises obtaining calcium imaging of the iPSC-vECs or the iPSC-CMs, or both.

In various embodiments, contacting the iPSC-CMs, iPSC-vECs, or both, with the test agent comprises culturing the iPSC-CMs, iPSC-vECs, or both, in the presence of liver-conditioned media. In various embodiments, contacting the iPSC-CMs, iPSC-vECs, or both, with the test agent comprises further culturing the iPSC-CMs, iPSC-vECs, or both with an agent capable of activating Akt signaling. In various embodiments, contacting the iPSC-CMs, iPSC-vECs, or both, with the test agent comprises culturing the iPSC-CMs, iPSC-vECs, or both in the presence of culture media flowing through the device.

In various embodiments, iPSC-vECs and iPSC-CMs are patient specific and the method models patient-specific parameters.

### Method of Making the Devices

Various embodiments provide for a method of producing a device of the present invention as described herein, comprising: seeding induced pluripotent stem cell derived cardiac cells (iPSC-CCs) on one surface of the membrane in the device; and seeding induced pluripotent stem cell derived endothelial cells (iPSC-ECs) on the other surface of the membrane in the device.

Various embodiments provide for a method of producing a device of the present invention as described herein, comprising seeding induced pluripotent stem cell derived cardiac cells (iPSC-CCs) in one chamber in the device; and seeding induced pluripotent stem cell derived endothelial cells (iPSC-ECs) in the other chamber in the device.

Various embodiments provide for a method of producing any one of the devices of the present invention, the method comprising: seeding induced pluripotent stem cell derived cardiomyocytes (iPSC-CMs) on one surface of the membrane in the device; and seeding induced pluripotent stem cell derived vascular endothelial cells (iPSC-vECs) on the other surface of the membrane in the device.

Various embodiments provide for a method of producing any one of the devices of the present invention, the method comprising: seeding induced pluripotent stem cell derived cardiomyocytes (iPSC-CMs) in one chamber in the device; and seeding induced pluripotent stem cell derived vascular endothelial cells (iPSC-vECs) in the other chamber in the device.

In various embodiments, seeding iPSC-CMs, seeding iPSC-vECs, or both comprise seeding the iPSC-CMs on top of a gel or in a gel, seeding iPSC-vECs on top of a gel or in a gel.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Induced Pluripotent Stem Cell-Derived Cardiomyocytes (iPSCs-CMs)

### Cardiac Differentiation of Human iPSC

Note: All media should be at least at RT when added.

After dissociation with EDTA or 1x cell detachment solution, seed approximately 100,000 human iPSCs on ECMS coated 6-well culture plates for differentiation (same steps as cell passaging procedures). When the cells reach 85% confluency, change the medium to RPMI/B27 without insulin medium with 6 µM GSK3-beta inhibitor CHIR99021 (CHIR) and maintain for 48 hr.

After 48 hr, replace the CHIR-containing culture medium with RPMI/B27 without insulin medium and leave alone for 24 hr (until day 3).

At day 3, change the media to RPMI/B27 without insulin with 5 µM Wnt inhibitor IWR1 and maintain for 48 hr (until day 5). NOTE: Wnt inhibition can also be attempted using other small molecule compounds, as described in earlier studies. IWR1 was selected over other small molecule Wnt inhibitors due to the increased range in which it has been shown to be effective in inhibiting Wnt signaling.

At day 5, change the medium back to RPMI/B27 without insulin medium and leave for 48 hr (until day 7).

At day 7, replace the medium with RPMI/B27 medium (with insulin) and replace medium every 3 days thereafter with the same medium. Spontaneous beating of cardiomyocytes should first be visible at approximately day 8 to day 10.

### Purification of Human Cardiomyocytes Through Glucose Starvation

At day 10 post-differentiation, change the medium in each well of the 6-well plate to 2 ml low glucose medium and maintain the cells in this medium for 3 days (until day 13).

At day 13, return cells to RPMI/B27 medium (with insulin). Optionally, replate cardiomyocytes prior to the second round of glucose starvation to help loosen non-cardiomyocytes from the culture plate, allowing for easier dissociation of non-cardiomyocytes during glucose starvation.
At day 13, aspirate medium, wash once with PBS, and dissociate the cells into single cells using 500 µL of cell disassociation enzyme for 5 min at 37° C. Specifically, after 5 min of enzyme treatment, use a 1,000 µl pipette to manually dissociate cardiomyocytes from the 6-well plate by repeatedly pulling up the cell disassociation enzyme and spraying it against the cardiomyocyte monolayer. Up to 30 pipetting repetitions may be required to dissociate the cardiomyocytes into single cells.
After cells are dissociated and are in single-cell form, collect all cells into a 15 ml conical tube filled with 5 ml of RPMI/B27 medium with insulin to dilute out the cell disassociation enzyme and centrifuge for 4 min at 200 x g. Aspirate and discard the supernatant.
Re-suspend the cells with 2 ml RPMI/B27 medium and plate onto a new ECMS-coated 6-well plate. Typically, higher confluency of cardiomyocytes helps with cell survival during replating. Aim to replate 2 million cells per new 6-well dish for optimal survival during replating.

At day 14, change the medium back to 2 ml of low glucose medium for a second glucose deprivation cycle. Culture the cells in this low glucose state for 3 more days. Most of the non-cardiomyocytes will die in this low-glucose culture condition.

At day 17, change the medium to 2 ml of RPMI/B27 medium with insulin. The remaining cells will be highly purified cardiomyocytes. These cardiomyocytes can be used for gene expression analysis, drug screening, metabolic analysis, and various other downstream assays.

Further information is found in, Sharma, A., Li, G., Rajarajan, K., Hamaguchi, R., Burridge, P.W., and Wu, S.M. (2015). Derivation of highly purified cardiomyocytes from human induced pluripotent stem cells using small molecule-modulated differentiation and subsequent glucose starvation. Journal of visualized experiments: JoVE., which is fully incorporated by reference herein.

### Example 2

### Induced Pluripotent Stem Cell-Derived Endothelial Cells (iPSCs-ECs)

In another example, iECs can be made by culturing (iPSCs) in the presence of CHIR99012 for about 2 days to generate mesoderm, culturing mesoderm in the presence of BMP4, VEGF, and FGF2 for about 2 days to generate vascular progenitor cells, culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In various embodiments, the vascular progenitors are cultured in the presence of EGM-MV3 and VEGF, and passages 2, 3, 4 or more times to generate endothelial cells. For example, iECs can express key markers such as CD31, CD34, VEGF, and VEGFA. Using a combination of growth factors, the Inventors were able to successfully produce endothelial cell types. Based on the described protocols, it appears that endothelial markers are more and purely expressed in Day 20 compared to Day 10 of differentiation -> time for maturation. Differentiation to be confirmed with other experiments: Dil-ac-LDL uptake, and TEER (resistance).

Alternatively, one can generate a quantity of endothelial cells made by a method of generating endothelial cells, including culturing (iPSCs) in the presence of CHIR99012 for about 2 days to generate mesoderm, culturing mesoderm in the presence of BMP4, VEGF, and FGF2 for about 2 days to generate vascular progenitor cells, culturing vascular progenitors in the presence of EGM-MV2 and VEGF for about 4-6 days to generate endothelial progenitor cells, and culturing endothelial progenitor cells in the presence of EGM-MV2 and VEGF to generate endothelial cells. In other embodiments, the vascular progenitor cells express one or more of: CD31+, CD34+, VEGF+, and VEGFA+ at day 20.

Further information is found in U.S. App. No. 15/458,185, 15/352,289, PCT App. No. PCT App. No. PCT/US2017/49115, PCT App. No. PCT/US2017/49193, PCT App. No. PCT/US2017/16079, PCT App. No. PCT/US2017/16098, PCT App. No. PCT/US2017/16079, PCT App. No. PCT/US2017/16098, PCT App. No. PCT/US2017/016098, PCT App. No. PCT/US2017/16079, PCT App. No. PCT/US2018-022511, PCT App. No. PCT/US2016/57724, and PCT App. No. PCT/US2017/49115, PCT App. No. PCT/US2019/023749, in U.S. App. No. 62/243,642, 62/277,723, 62/332,727, and 62/380,780, which are fully incorporated by reference herein.

Methods to convert human induced pluripotent stem cells (hiPSCs) to cardiomyocytes (hiPSC-CMs) and endothelial cells (hiPSC-ECs) have enabled cardiovascular cells to be mass-produced in vitro for disease modeling and drug screening. The hiPSC-CMs express most ion channels and sarcomeric proteins found in adult human CMs and can spontaneously contract. Similarly, hiPSC-ECs can uptake LDL and undergo angiogenic sprouting. These hiPSC-CMs and hiPSC-ECs can be made in approximately two weeks using defined differentiation protocols, and they can be genetically customized using genome editing technologies such as CRISPR/Cas9. Additionally, hiPSC-derived CMs and ECs can recapitulate, at the cellular level, phenotypes for cardiovascular diseases including viral myocarditis, Brugada syndrome, long-QT syndrome, dilated cardiomyopathy, hypertrophic cardiomyopathy, chemotherapy-induced cardiomyopathy, and congenital heart disease. The hiPSC-CMs are also responsive to inotropic drugs such as norepinephrine, and their beating rates can be controlled via electrical stimulation. Because hiPSC-CMs and hiPSC-ECs can be purified and replated for downstream applications, research groups in academia and industry have started utilizing these cells as a complementary platform to non-human and non-cardiovascular model systems, for high-content imaging and drug screening assays.

The Inventors have previously conducted high-throughput screening of TKI-induced cardiomyocyte toxicity using patient-specific hiPSC- CMs. Screening multiple classes of TKIs, the Inventors demonstrated that TKIs caused significant toxicity on hiPSC-CMs, measured via various functional assays. The hiPSC-CMs treated with TKIs with FDA black-box warnings (e.g., nilotinib and vandetanib) displayed contractile dysfunction at clinically-relevant concentrations. Contractility analysis of beating cells and whole cell patch- clamping revealed alterations in hiPSC-CM function starting within hours of TKI treatment. Ca2+ imaging demonstrated irregular Ca2+ transients in TKI-treated hiPSC-CMs, suggesting that TKI-induced dysfunction in hiPSC-CMs manifests in altered Ca2+ cycling. Using these functional readouts, the Inventors established a “cardiac safety index” ranking cardiotoxic TKIs. The Inventors reversed TKI-induced CM toxicity by co-treatment with Akt pathway activators IGF and insulin. Sharma et al Science Translational Medicine 2017 and Sharma et al Nature Protocols 2018 demonstrated that hiPSC-CMs can be used to screen TKIs for cardiotoxicity, which is fully incorporated by reference herein.

However, 1) hiPSC-EC vasculotoxicity and CM/EC interaction in an integrated system during TKI treatment was not examined. 2) hiPSC-CM data was obtained from immature 2D mono-cultured cells 3) TKIs were not metabolized in a liver system before adding on hiPSC- CMs. 4) Only FDA-approved TKIs were evaluated for toxicity. 5) Although VPTKIs were highly cardiotoxic on hiPSC-CMs, the study did not exclusively focus on VPTKI-TOX, which exhibits a significant endothelial toxicity component. These items are addressed by the present invention described herein.

### Example 3

### Employing Cardiovascular Organ-Chips to Evaluate the Molecular Mechanisms Driving VPTKI-TOX and Develop Approaches For Cardioprotection

The model the Inventors have allows the accurate identification of cardiotoxic and vasculotoxic VPTKIs in a safe, preclinical setting. The Inventors utilize organ-chip technologies, which utilize a microfluidic architecture to enable the 3D co-culture of distinct cell types. Microfluidic components enable a shared “systemic circulation”, by which cell culture media and nutrients can flow between multiple cell types on the integrated chip. Organ-chip systems enable modeling of multicellular interactions, tissue-tissue interfaces, mechanical forces and shear stress from vascular perfusion, and physiochemical microenvironments.

The Inventors use microfluidic organ chips exemplified by a microengineered organ-chip, this example including includes two fluidically-independent channels separated by a porous membrane that allows for cell-cell interaction and diffusion similar to the in vivo setting. Cells in each channel can be seeded with extracellular matrix and maintained either in static culture or connected to a system which provides continuous flow of cell culture media.

One can deploy such organ chip designs to investigation VPTKI-TOX by seeding hiPSC-derived vascular ECs and hiPSC- CMs to decipher cellular interaction and cross-talk. Such an approach allows exploration of 1) enhance hiPSC-CM maturation via shear stress from media flow 2) enable examination of VPTKI-TOX mechanisms on ECs and CMs simultaneously 3) model VPTKI liver metabolism by adding VPTKIs to liver cells and adding liver-conditioned media to the organ chip 4) enable examination of cardioprotective mechanisms against VPTKIs simultaneously on CMs and ECs.

### Example 4

### Develop and Characterize a Multi-Lineage, hiPSC-Derived, Cardiovascular Cell Culture Platform on a Fully-Integrated, Microfluidic Organ-Chip (“Organ Chip”)

Initial studies rely on organ-on-chip platform, harboring wild type hiPSC-CMs/ECs. The Inventors focus on these cells since VPTKIs exhibit dual myocardial (e.g., heart failure, QT-interval prolongation) and vascular (e.g., hypertension) toxicities. The Inventors use 5 healthy control hiPSC lines to test organ chip reproducibility.

As described, one can seed of hiPSC-CMs/ECs in separate channels of organ chips to investigate cellular interactions and cross-talk. The Inventors uses Matrigel® as extracellular matrix for EC/CM culture. Data show hiPSC-CMs can be grown on organ chip surfaces. Immunofluorescence for EC (e.g., CD144) and CM (e.g., cTnT) markers confirms cell identity. Measurement of trans-endothelial electrical resistance (TEER) confirms hiPSC-EC function and integrity of the vascular EC channel on the organ chip. TEER is an accepted quantitative technique to measure tight junction integrity in EC monolayers. Calcium imaging and patch- clamp electrophysiology on organ chip CMs is performed and are standard for confirming hiPSC-CM function.

Use fluorescent reporter hiPSC-CMs and sarcomere contractility analysis as a real-time readout of hiPSC-CM functional maturation on organ chips. Data suggests that microfluidic organ chips can enhance maturity of hiPSC-derived cells. Also, shear stress from fluid flow enhances hiPSC-CM maturity. Mature hiPSC- CMs are needed to accurately evaluate VPTKI-TOX, and novel approaches to induce hiPSC-CM maturation are desired in general. The Inventors believe that culture on the organ chip enhances CM maturity. the Inventors recently developed fluorescent sarcomere reporter hiPSC-CMs (Sharma et al 2018, Curr Protoc Hum Gen) for use in a high-throughput sarcomere analysis platform, “SarcTrack”, to assess sarcomere contractility and alignment as a function of cell maturity. The Inventors will use TTN-GFP hiPSC-CMs and SarcTrack to assess hiPSC-CM maturation on organ chips.

### Isolate Organ Chip hiPSC-CMs and Analyze Transcriptional Maturation by Single-Cell Expression

To mechanistically confirm transcriptional maturation of hiPSC-CMs on the organ chip, the Inventors conduct single- cell gene expression analysis using the 10X Genomics Chromium system in the Svendsen lab. Specifically, the Inventors examine the expression of CM sarcomeric maturation markers (e.g., higher expression of MYH7 over MYH6) and shift from glycolytic to fatty acid metabolism. The Inventors benchmark organ chip hiPSC-CMs against 2D mono-cultured hiPSC-CMs and adult human CMs as a control.

### Demonstrate Reproducibility of Organ Chip by Producing Organ Chips From 5 Independent Control hiPSC Lines

Line-to-line variability can be an issue for hiPSC differentiation. To show the fidelity of The Inventors’ system, the Inventors seed organ chips with hiPSC-CMs and hiPSC-ECs from 5 independent, healthy control hiPSC lines. The Inventors use blood/Sendai-virus reprogrammed, de-identified, healthy control hiPSC lines. The Inventors expect the CMs and ECs to adhere to independent channels of the organ chip, function normally, and express standard CM/EC marker genes, with hiPSC-CMs exhibiting enhanced maturity. The Inventors make organ chips from 5 independent hiPSC lines to demonstrate reproducibility.

In other embodiments, one can utilize transwell membranes could be used to concurrently grow CMs/ECs. Additionally, the Inventors explore hiPSC-vascular smooth muscle cell and fibroblast differentiation, since VPTKI-TOX may also be driven by these cell types.

### Example 5

### Subject the Developed Organ Chip to Liver-Metabolized VPTKIs to Examine Cellular and Molecular Mechanisms Driving VPTKI Cardiotoxicity

One can assess VPTKI-TOX on organ chips after VPTKIs undergo 24-hour liver metabolism in human hepatocytes. The Inventors previously examined TKI toxicity in 2D-monocultured hiPSC-CMs. However, TKIs were not pre-metabolized on a liver system and were not exposed to hiPSC- CMs/ECs concurrently. This presents a more physiologically-relevant model for assaying VPTKI-TOX.

### Model TKI Metabolism by Adding VPTKIs to Liver Cells & Adding Liver-Conditioned Media

The majority of small molecule TKIs undergo liver metabolism, but this is not addressed in current models of TKI cardiotoxicity. Metabolic activation (bioactivation) of TKIs by cytochrome P450 (CYP) enzymes leading to formation of biochemically active products is a key initiator of TKI-induced hepatotoxicity and cardiotoxicity. Several VPTKIs undergo bioactivation to form active metabolites, including axitinib, ponatinib, sunitinib, and some investigational TKIs. CYP3A metabolism catalyzes drug elimination for most small molecule TKIs.

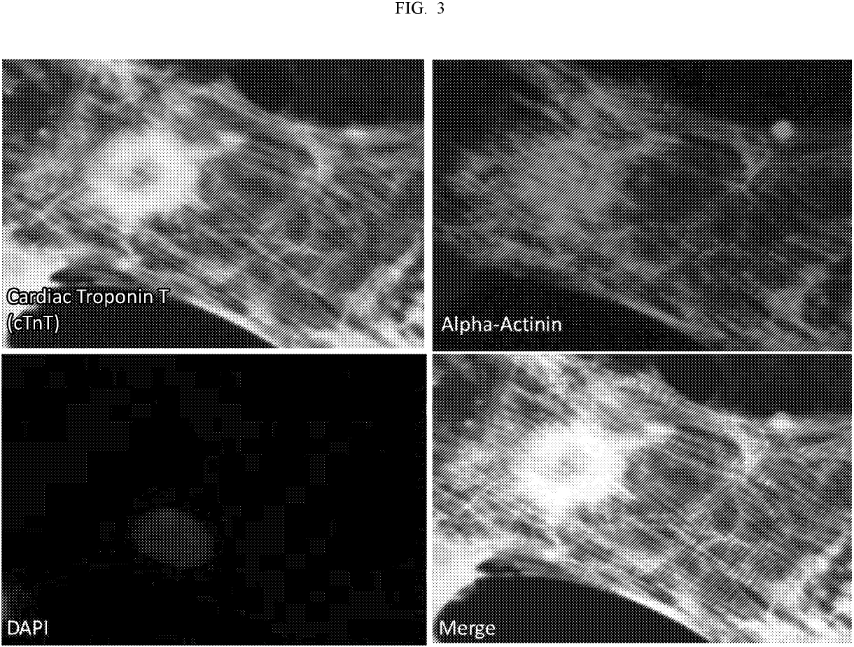

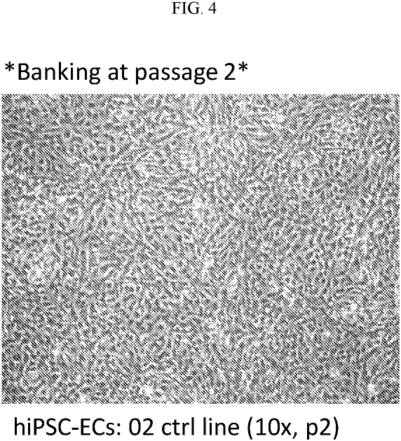

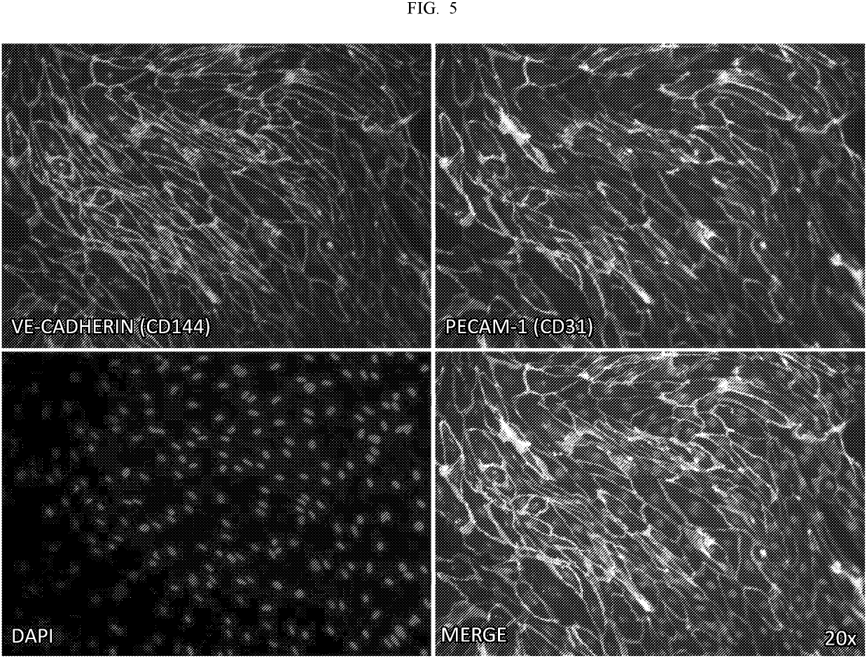

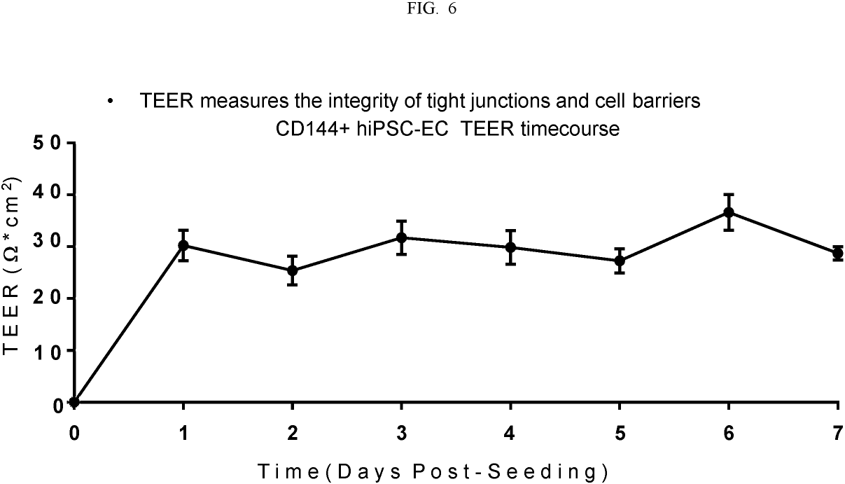

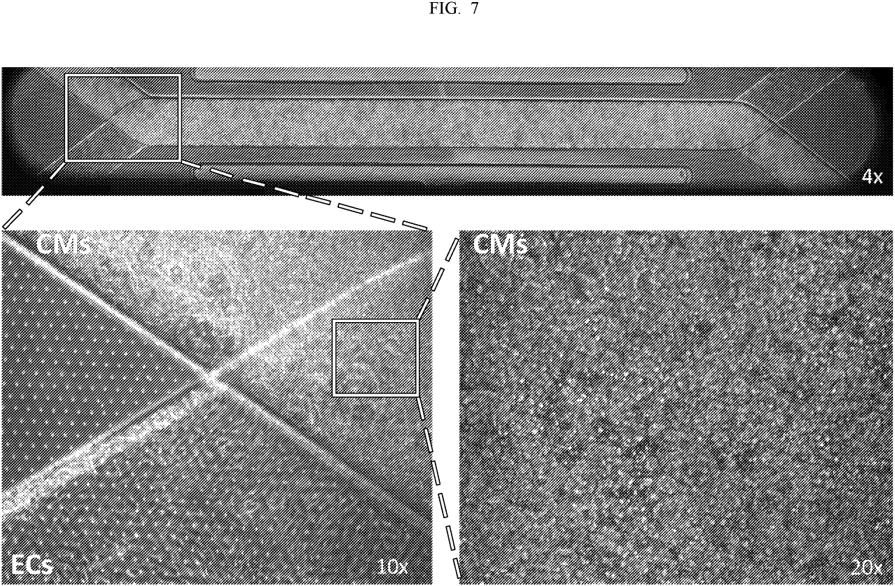

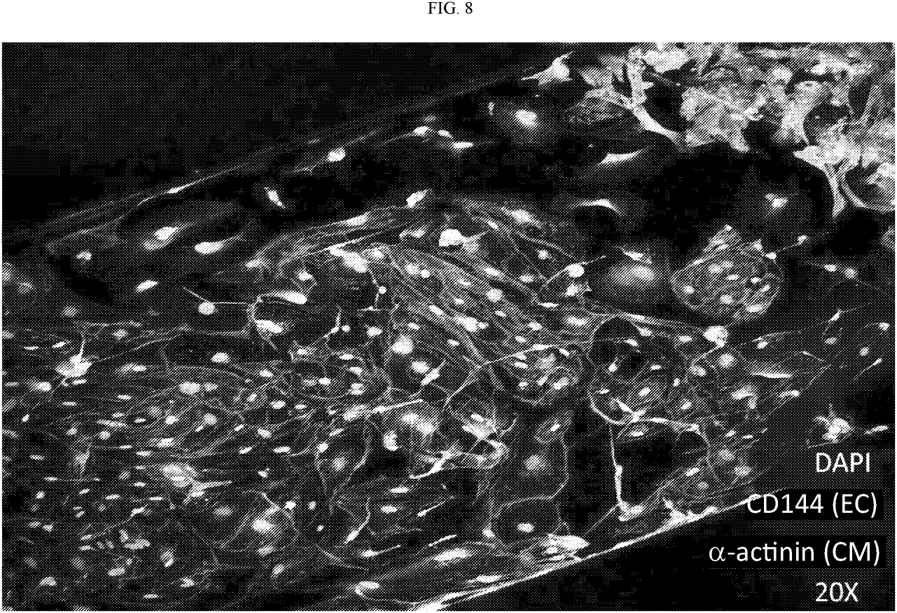

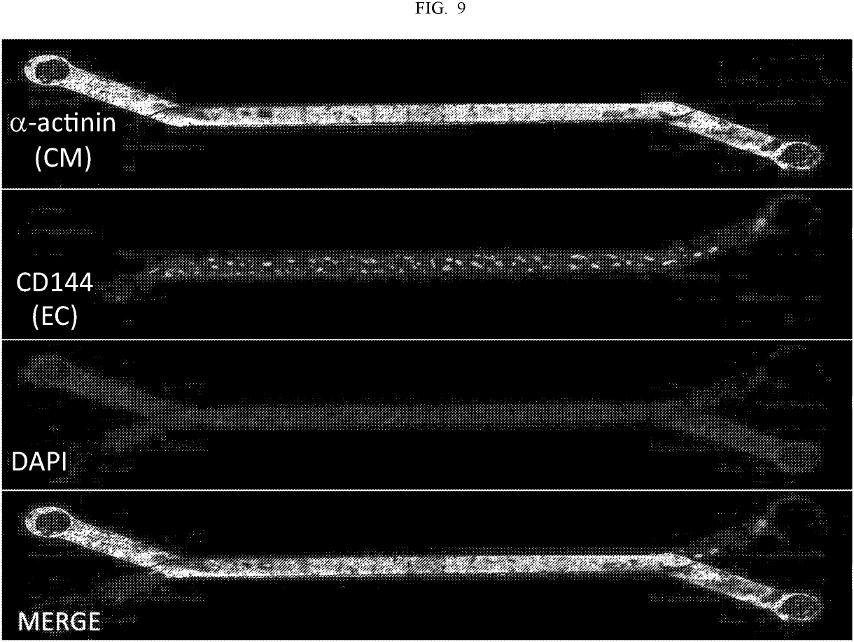

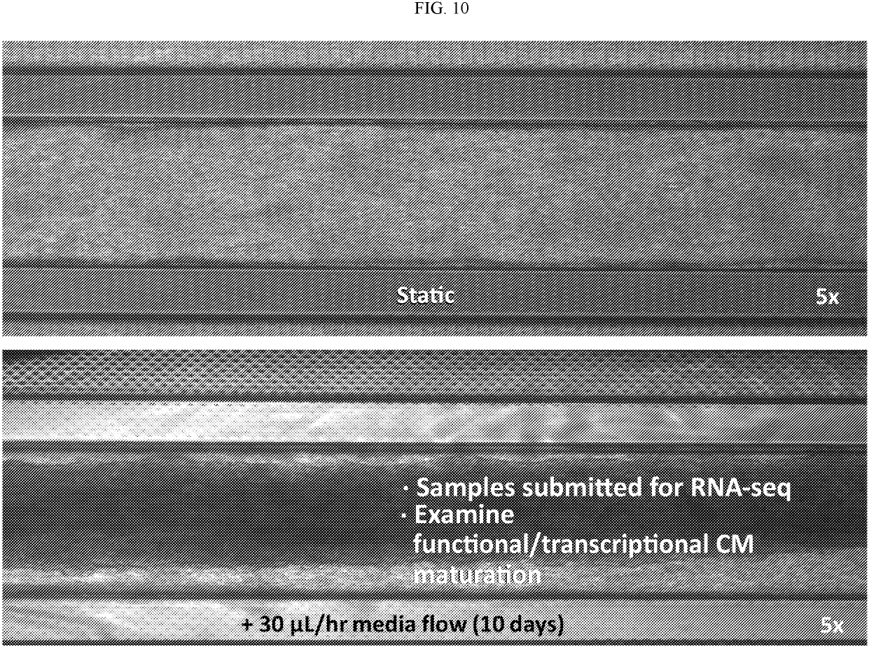

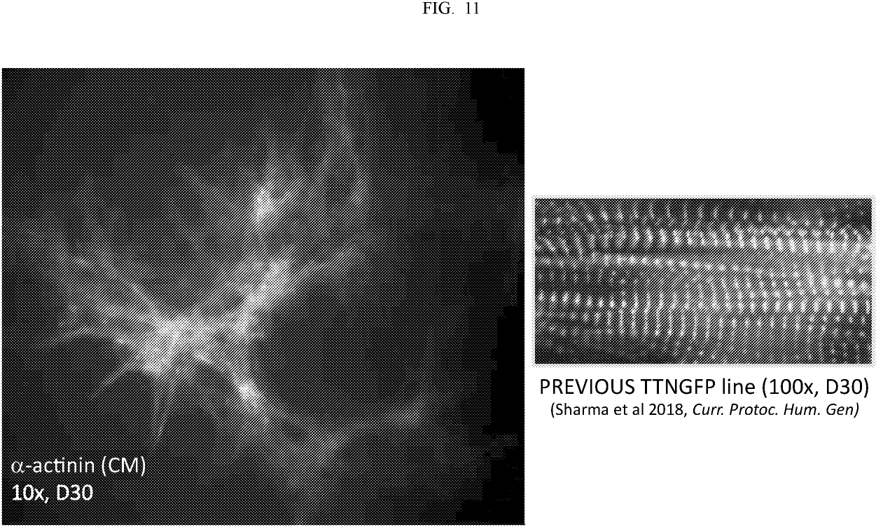

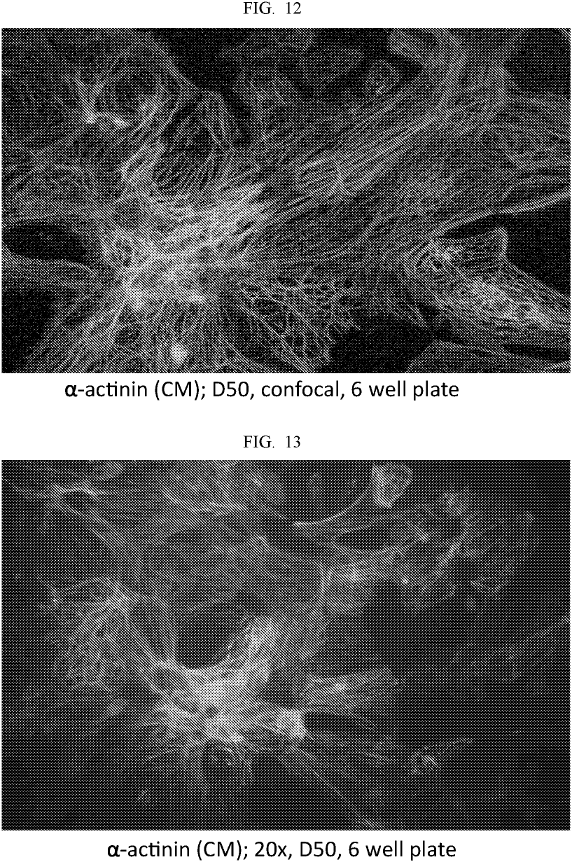

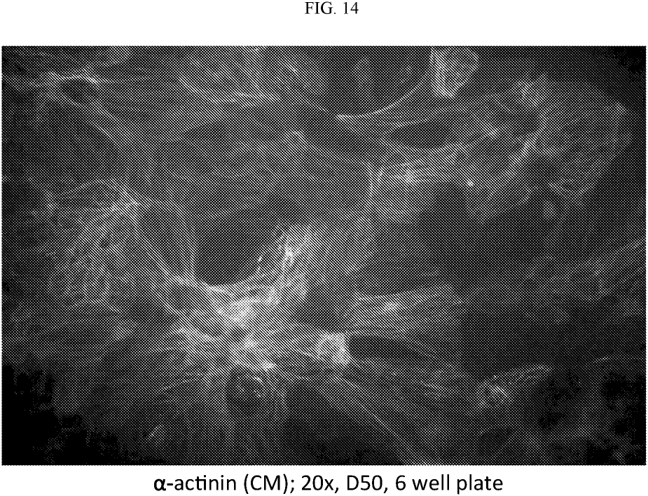

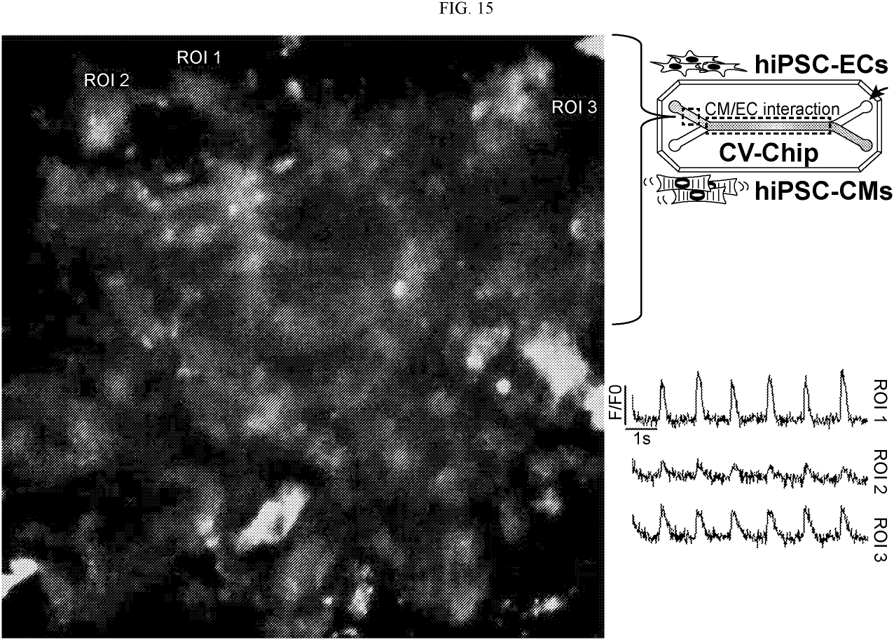

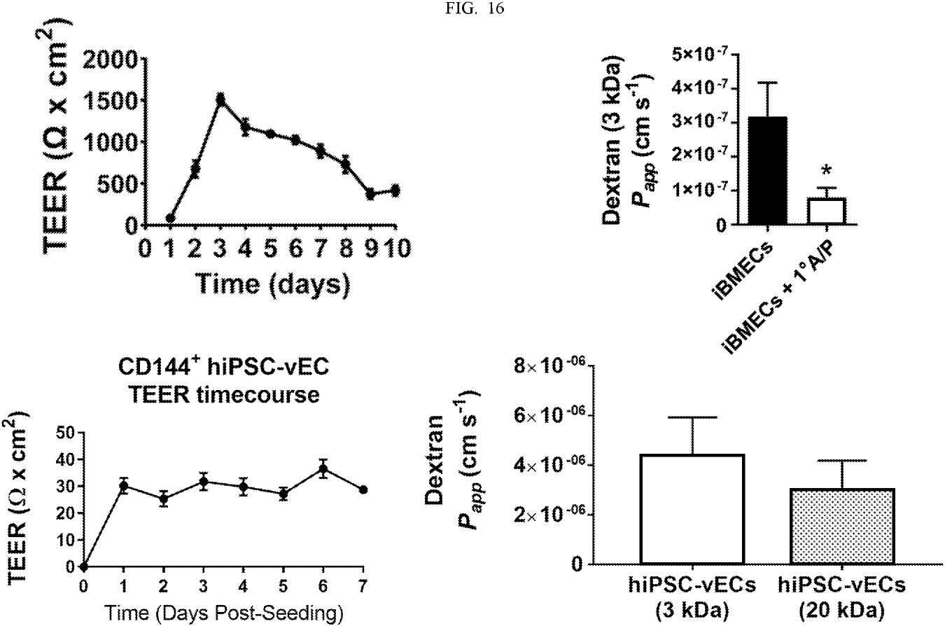

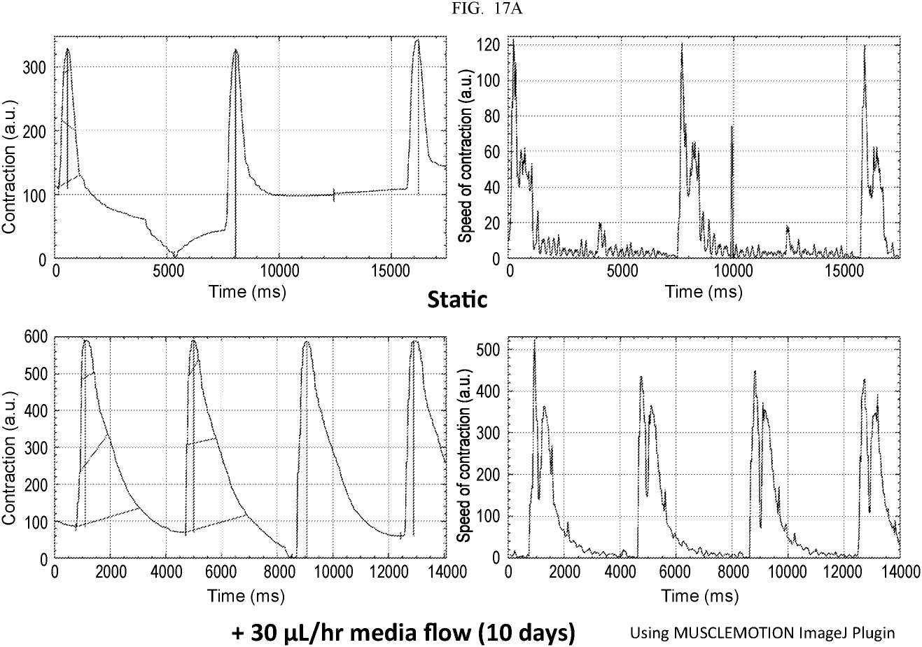

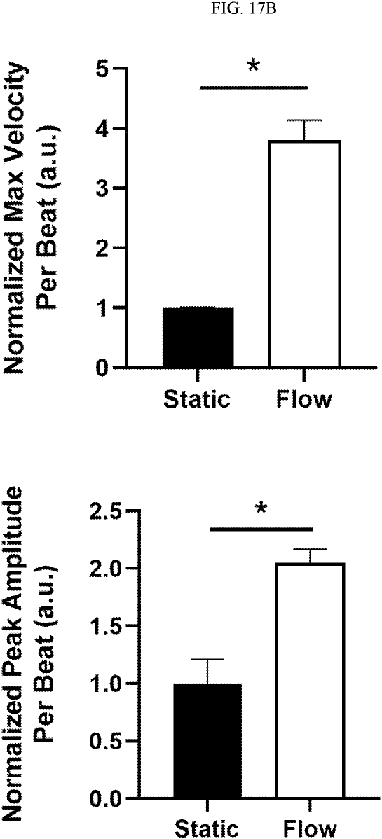

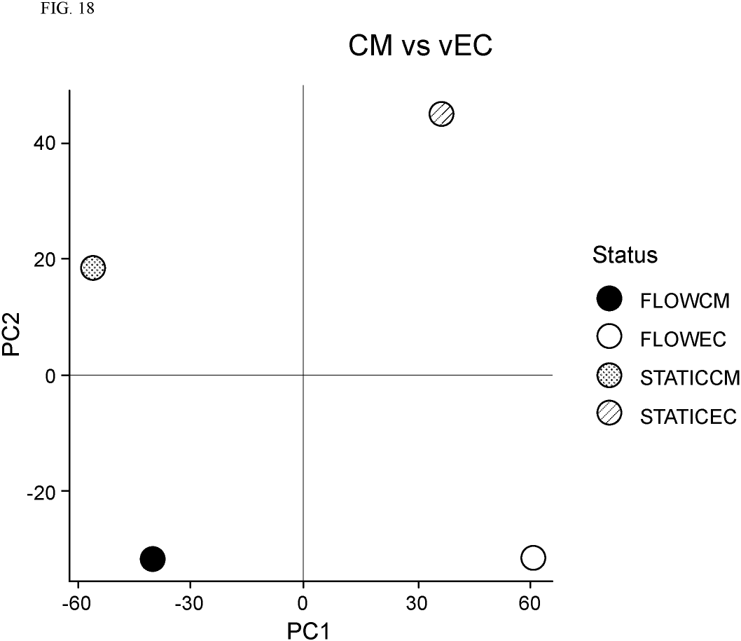

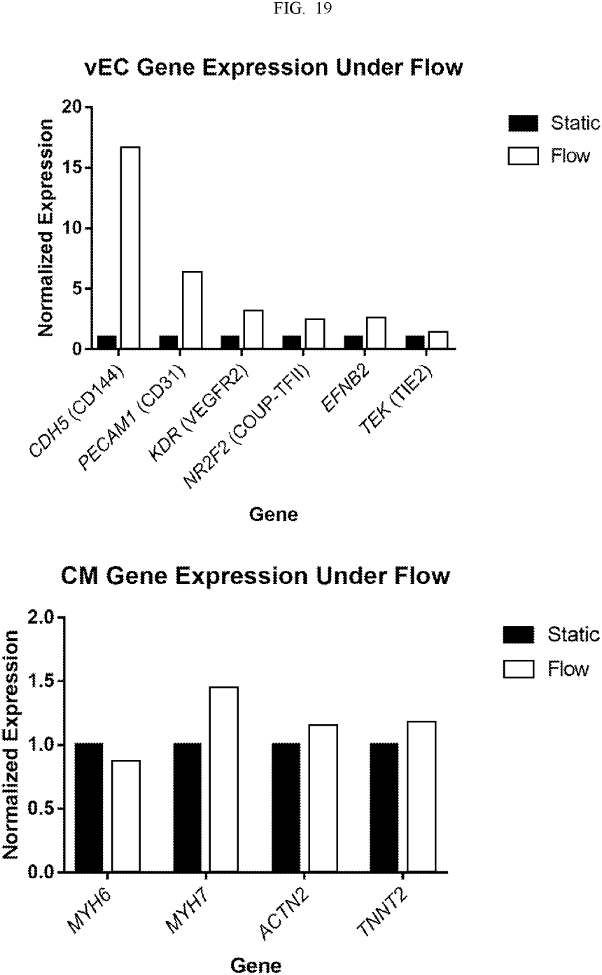

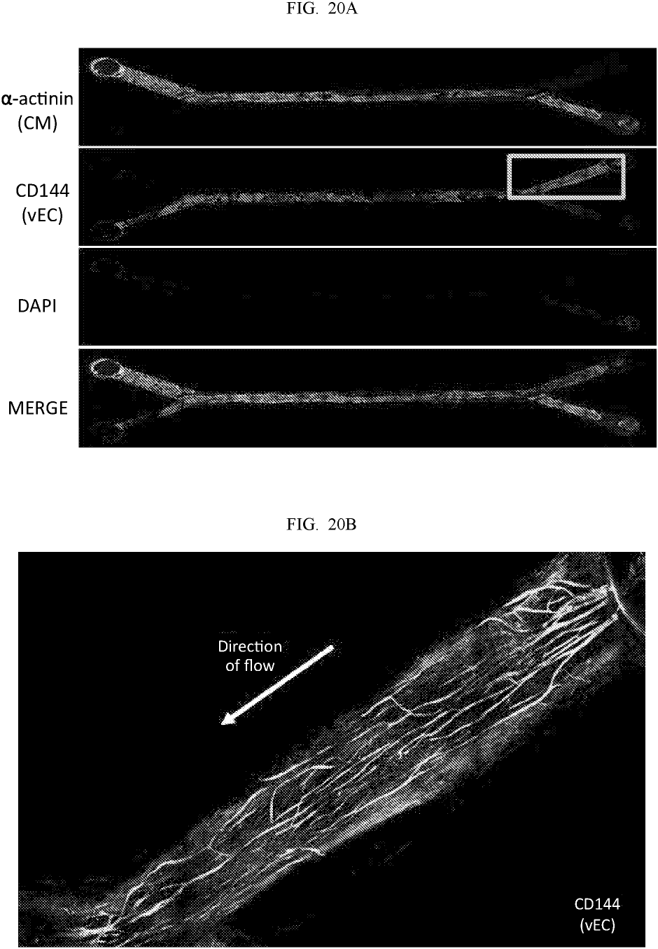

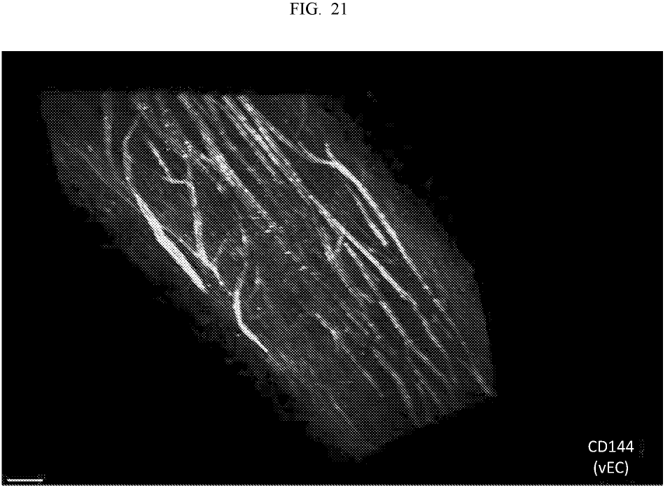

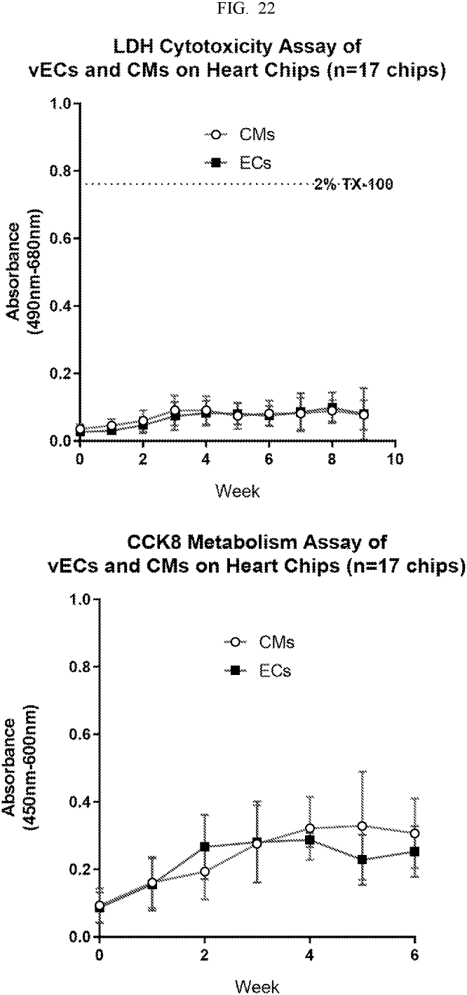

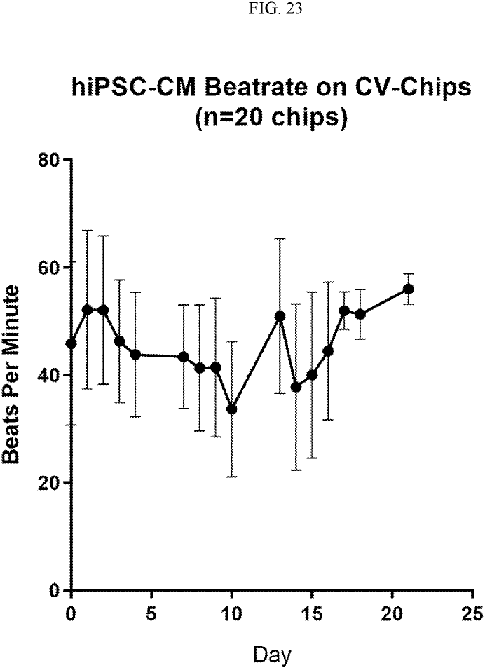

CYP3A enzymes catalyze TKI bioactivation, but human liver cell lines (e.g., HepG2) do not highly express CYP3A. Thus, to model VPTKI liver metabolism, the Inventors will use HepG2-CYP3A4 human hepatocytes (Hera Bio Labs), stably overexpressing CYP3A4 (FIG. 3). HepG2-CYP3A4 cells will be grown in the same culture medium. The Inventors will subject HepG2-CYP3A4 cells to 24 hr of 0-10 µM VPTKIs (e.g., sorafenib, sunitinib). PrestoBlue cell viability assay will quantify VPTKI-induced hepatotoxicity. Mass spectrometry will confirm bioactive metabolites resulting from VPTKI bioactivation by HepG2-CYP3A4 cells. Finally, the Inventors add liver cell-conditioned media from the LD50 dose associated with VPTKIs to organ chips for 24 hrs. As a control, the Inventors add media (containing the same VPTKIs) conditioned on HEK293 cells to organ chips for 24 hrs. PrestoBlue will compare viability of organ chips treated with HEK293 media containing un-metabolized TKIs to liver-conditioned media containing liver-metabolized TKIs.

### Subject Organ Chips to Known Cardiotoxic VPTKIs and Next-Generation VPTKIs and Evaluate hiPSC-CM/EC Cell Type-Specific Functional Toxicities With Sarcomere Contractility, TEER, and Cytotoxicity Analyses

Using the established organ chip, the Inventors examine VPTKI-TOX of known cardiotoxic VPTKIs and next-generation VPTKIs under development. The Inventors test sunitinib and sorafenib, well-studied VEGFR1/2/3 and PDGFR inhibitors that decrease survival in the zebrafish model and cause hypertension, ischemia, and cardiac side-effects in patients. Also, the Inventors assess other VPTKIs such as pazopanib, vandetanib, axitinib, ponatinib, regorafenib, and lenvatinib. Ponatinib in particular has severe vascular toxicity and a hepatotoxicity-associated FDA black-box warning. VPTKIs in development, cediranib and tivozanib, are evaluated. EGFR inhibitor gefitinib typically does not exhibit cardiotoxic side effects and will be used as a non-toxic control.

### VPTKI Dosing/Timing

The Inventors established TKI concentrations for assessment of toxicity at 0-100 µM. An optimal time-point is 24-72 hours, similar to experiments in rat myocytes. (ii) Cell imaging. Cell death will be assessed at 24-96 hours after exposure to VPTKIs. Live cells are stained for Calcein AM and Hoechst dye. (iii) Cell viability and apoptosis. The Inventors use flow cytometry for annexin V (early apoptosis) and PrestoBlue, a colorimetric assay for measuring viability. (iv) Alteration in kinome. The Inventors use high-throughput assessment of alterations in CM/EC receptor tyrosine kinase (RTK) phosphorylation after TKI treatment (v) Functional readouts. The Inventors use readouts for altered hiPSC-CM function (sarcomere contractility analysis, calcium imaging, patch clamp electrophysiology) and hiPSC-EC function.

### Elucidate Mechanisms of VPTKI-Cardiotoxicity via RNA-Sequencing, Proteomics, and Metabolomics

The Inventors perform in-depth analysis of VPTKI-treated CV-Chips using single-cell RNA-seq of patient-specific hiPSC-CMs/ECs, at baseline and under TKI stress, using a 10x Genomics Chromium system. Sequencing data are mapped to the reference human genome (hg38). Differentially expressed genes will be determined using the Seurat algorithm. Based on The Inventors’ toxicity data, the Inventors specifically direct analysis towards genes correlated with GO terms that are associated with cardiovascular toxicity or biology. HiPSC-CM RNA-seq data suggests minimal changes in gene expression occur at 0.1 µM VPTKI treatment, activation of preconditioning and development-regulatory gene expression occurs at 1 µM, and apoptosis genes predominate at 10 µM. Thus, the Inventors conduct analysis on samples treated with 0 µM and 1 µM of VPTKIs for 24 hr. Proteomics and metabolomics analyses are conducted using an OrbiTrap Elite mass spectrometer at low-doses of VPTKI to 1) confirm phosphorylation changes in TKI-treated CMs/ECs 2) identify novel metabolites that may serve as biomarkers for VPTKI-TOX 3) confirm metabolism of VPTKIs by HepG2-CYP3A4 cells.

The Inventors subject cells to liver-metabolized VPTKIs and assess cardiotoxicity on hiPSC-CMs/ECs. The Inventors anticipate that liver-metabolized VPTKIs elicit a different cytotoxicity profile on organ chips than reported previously in isolated CMs. The Inventors believe that this system more accurately models in vivo drug metabolism with VPTKIs, especially since the majority of VPTKIs are metabolized in the liver. Through transcriptomic and proteomic analysis, the Inventors anticipate identification of novel biomarkers for VPTKI-TOX that may enable clinicians to modify VPTKI treatment strategies accordingly.

### Example 6

### Activate Akt Signaling to Alleviate VPTKI-TOX in Patient-Specific Organ Chips

Data suggests VPTKI treatment inhibits CM viability and function. Thus, finding means by which cardiovascular cells can be protected from VPTKIs is of critical interest. VPTKI treatment on hiPSC-CMs was shown to activate compensatory anti-apoptotic Akt signaling, augmented via exogenous IGF or insulin. The Inventors extend this work to the organ chip and examine how Akt-activation may protect cardiovascular cells from VPTKI-TOX.

Hyperactivation of Akt pathway in healthy control CV- Chips to phenocopy cardioprotective mechanisms against VPTKIs. Using the organ chip, the Inventors first investigate molecules that will enhance CM/EC survival independent of VPTKI treatment. These molecules likely upregulate PI3K/Akt signaling in hiPSC-ECs and CMs, as PI3K/Akt is a known pro-survival pathway in CMs and ECs. However, small molecule activators of PI3K/Akt are not as common or effective as growth factors, but they have the advantage of being less costly for screening. The Inventors test candidate small molecule Akt activators including SC79. Candidate Akt-activating growth factors will include vascular endothelial growth factor (VEGF) which has well-established protective roles in vascular function, and insulin growth factor (IGF) which the Inventors have identified as upregulating cardioprotective signaling in hiPSC- CMs. In other cell types, VEGF and IGF upregulate anti-apoptotic PI3K/Akt and Erk signaling. However, the roles of these molecules are not well-understood in co-cultured CMs and ECs. The Inventors use kinome screening to confirm in organ chips that VEGF and IGF can upregulate Akt phospho-signaling. The Inventors treat organ chips with concentrations of growth factors as determined by The Inventors’ previous study. The Inventors will also develop a lentiviral Akt overexpression vector that will enable stable Akt hyperactivation in both hiPSC-CMs and ECs, to stimulate cardioprotection.

### Generate Patient-Specific Organ Chips From VPTKI-TOX Individuals and Controls to Recapitulate Toxicities When Subjected to VPTKIs

Attempt to alleviate cardiotoxicity by Akt pathway stimulation. Based on study inclusion/exclusion criteria, the Inventors identify 5 patients with heart failure (HF) or hypertension from VPTKI-TOX, 5 with non-VPTKI-induced HF or hypertension, and 5 controls. Subsequently, the Inventors generate hiPSCs from these 15 individuals and create CV- Chips. Next, the Inventors examine patient-specific differences in VPTKI-TOX across the patient-specific CV- Chips. Previous data suggests that VPTKIs exhibit high levels of cytotoxicity on cardiovascular cell types. For example, the Inventors determined that ponatinib, labeled with an FDA black box warning for vasculotoxicity, was the most cytotoxic of all TKIs studied in The Inventors’ previous hiPSC-CM study. Thus, the Inventors focus first on alleviating toxicity caused by these VPTKIs, which include sorafenib, ponatinib, and sunitinib. The Inventors treat organ chips with VPTKIs for 0-72 hours at 0-10 µM concentration, and co-treat candidate protective factors VEGF and IGF at concentrations listed in prior literature. Since VPTKIs target the VEGFR2 signaling pathway, a critical survival pathway in ECs, the Inventors believe that exogenous introduction of VEGF concurrently during VPTKI treatment may protect patient-specific hiPSC-ECs from VPTKI-TOX. To assess alleviation of cardiovascular toxicity, the Inventors examine CM/EC cell morphology via immunofluorescence, reactive oxygen species production, and signs of apoptosis using assays described herein. Additionally, to gain greater mechanistic understanding of how these protective compounds are functioning, the Inventors conduct proteomic phosphorylation analysis, which will determine if VEGF or IGF is hyperactivating protective pathways other than PI3K/Akt (e.g., MEK/Erk) to promote organ chip cell survival and counteract VPTKI-mediated VEGFR2 inhibition. The Inventors will also assess the restoration of CM/EC function after protective molecule treatment, using functional assays described.

The Inventors use Akt signaling pathway hyperactivation to confirm the protective properties of compounds that may shield organ chips from cardiovascular toxicity, with a focus on identifying additional compounds associated with simultaneous vasculoprotection and cardioprotection. The Inventors have expertise with these procedures from The Inventors’ previous study, and the Inventors have candidate molecules selected, including VEGF/IGF. The Inventors’ focus is on the PI3K/Akt pathway as it is a well-established cellular anti- apoptosis pathway that can be coopted for protection against VPTKIs. For example, in The Inventors’ previous study, the Inventors demonstrated that insulin and IGF treatment significantly enhanced PI3K/Akt phosphorylation to promote cardioprotection in hiPSC-CMs. However, the Inventors also examine the upregulation of the Erk signaling pathway, another conserved anti-apoptotic signaling pathway.

The Inventors examine organ chips to determine if activation of Akt signaling protects patient-specific hiPSC-ECs and hiPSC-CMs from VPTKI-TOX. The Inventors evaluate whether protective compounds shield cells from death or, alternatively, induce proliferation. These results appear similar on the PrestoBlue assay. To differentiate, the Inventors perform cell counting prior to the assay, which will determine if cell numbers are increasing (indicating proliferation) after adding Akt activators, or if cell numbers stay constant (indicating survival without proliferation). Also, IGF/Insulin could rescue cancer cells while protecting CV cells. The Inventors explore non-growth factor approaches to activate Akt signaling, and also CV-specific targeting via cardiotropic AAV expression.

Cardiotoxicity affects >10% of patients undergoing TKI chemotherapy. Hypertension is a major side-effect of VPTKIs in particular. However, it remains difficult to accurately identify cardiotoxic VPTKIs during preclinical development.

As described, the Inventors develop and evaluate of a multi-lineage, integrated cardiovascular organ-chip to provide a significant resource for the study of VPTKI- TOX. VPTKI metabolism in liver cells followed by detailed analysis of VPTKI-treated organ chips using TEER, sarcomere contractility analysis, and cell viability in combination with -omics level analysis, will provide mechanistic insights and a better understanding of VPTKI-TOX. The -omics level analyses will be deposited and accessible to bioinformatics analysis by other groups. Cardioprotection against VPTKI-TOX will be explored using patient-specific hiPSCs. The concept that activation of Akt signaling can reverse VPTKI-TOX phenotypes has never been described before.

Additional representative embodiments include, but are not limited to the following.

A method, comprising: providing induced pluripotent stem cell derived cardiomyocytes (iPSC-CMs) and induced pluripotent stem cell derived endothelial cells (iPSC-ECs) in a fluidic device; contacting the iPSC-CMs, iPSC-ECs, or both, with one or more candidate agents; measuring one or more phenotypes of interest in the iPSC-CMs, iPSC-ECs, or both; selecting one or more candidate agents based on measurements of the one or more phenotypes of interest in the iPSC-CMs, iPSC-ECs, or both.

The method of a preceding paragraph, wherein the iPSC-CMs express a fluorescent reporter.

The method of a preceding paragraph, wherein one or more candidate agents is a VEGFR2/PDGFR-inhibiting tyrosine kinase inhibitor.

The method of a preceding paragraph, wherein measuring one or more phenotypes of interest comprises measuring fluorescence.

The method of a preceding paragraph, wherein measuring one or more phenotypes of interest comprises measuring sarcomere contractility.

The method of a preceding paragraph, wherein measuring one or more phenotypes of interest comprises measuring TEER resistance.

The method of a preceding paragraph, wherein measuring one or more phenotypes of interest comprises measuring expression levels of one or more of MYH7 and MYH6.

The method of a preceding paragraph, wherein measuring one or more phenotypes of interest comprises measuring cell viability.

The method of a preceding paragraph, wherein contacting the iPSC-CMs, iPSC-ECs, or both, with one or more candidate agents comprises culturing the iPSC-CMs, iPSC-ECs, or both, in the presence of liver-conditioned media.

The method of a preceding paragraph, wherein contacting the iPSC-CMs, iPSC-ECs, or both, with one or more candidate agents comprises further culturing the iPSC-CMs, iPSC-ECs, or both with an additional agent capable of activating Akt signaling.

The method of a preceding paragraph, wherein the fluidic device is a microfluidic device.

The method of a preceding paragraph, wherein the microfluidic device is an organ chip.

A method, comprising: providing fluidic device; seeding induced pluripotent stem cell derived cardiomyocytes (iPSC-CMs) in one or more chambers in the fluidic device; and seeding induced pluripotent stem cell derived endothelial cells (iPSC-ECs) in one or more chambers in the fluidic device.

An apparatus made by the method of the preceding paragraph.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A device, comprising:
a membrane comprising a top surface and a bottom surface;
a first channel in fluidic communication with the top surface of the membrane;
a second channel in fluidic communication with the bottom surface of the membrane, wherein the first and second channels each comprises a surface that is parallel to the membrane;
induced pluripotent stem cell derived-endothelial cells (iPSC-ECs) in the first channel or the second channel; and
induced pluripotent stem cell derived-cardiac cells (iPSC-CCs) in the first channel or the second channel,
wherein the iPSC-ECs and the iPSC-CCs are in different channels.

2. The device of claim 1, wherein
the iPSC-ECs are induced pluripotent stem cell derived-vascular endothelial cells (iPSC-vECs); and
the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs).

3. The device of claim 1, wherein
the iPSC-ECs are induced pluripotent stem cell derived-vascular endothelial cells (iPSC-vECs); and
the iPSC-CCs are induced pluripotent stem cell derived-cardiomyocytes (iPSC-CMs).

4. The device of claim 1,further comprising: at least one inlet port adapted for fluid entering the at least one inlet port; and at least one outlet port adapted for fluid exiting the at least one outlet port.

5. The device of claim 1, further comprising:
a top chamber; and
a bottom chamber,
wherein:
the membrane is between the top chamber and the bottom chamber;
the first channel is fluidically coupled to the top chamber; and
the second channel is fluidically coupled to the bottom chamber.

6. (canceled)

7. (canceled)

8. The device of claim 1, wherein the first and second channels comprise polydimethylciloxane.

9. The device of claim 1, wherein the iPSC-CCs are in the first channel and the iPSC-ECs are in the second channel.

10. The device of claim 3, wherein the iPSC-CMs are in the first channel and the iPSC-vECs are in the second channel.

11. The device of claim 1, wherein:
the first channel and the second channel are microfluidic channels;
the membrane comprises polydimethylciloxane;
iPSC-ECs and iPSC-CCs are patient specific;
the iPSC-CCs express a fluorescent reporter; and/or
the iPSC-ECs are alpha-actinin-GFP iPSCs, or the iPSC-CCs are alpha-actinin-GFP iPSC-CCs, or both.

12. An organ chip device, comprising:
induced pluripotent stem cell derived-endothelial cells (iPSC-ECs);
induced pluripotent stem cell derived-cardiac cells (iPSC-CCs); and
a membrane separating the iPSC-ECs and iPSC-CCs.

13. (canceled)

14. (canceled)

15. (canceled)

16. The device of claim 1, further comprising one or more gels and the iPSC-ECs or the iPSC-CCs, or both separately having been seeded on top of or into the one or more gels.

17. (canceled)

18. (canceled)

19. (canceled)

20. The device of claim 1, wherein;
the iPSC-ECs are human iPSC-ECs (hiPSC-ECs); and
the iPSC-CCs are human iPSC-CCs (hiPSC-CCs).

21. (canceled)

22. (canceled)

23. A method of assessing a test agent, comprising:
contacting the test agent to the device of claim 1;
measuring a parameter; and
assessing the test agent based on the measured parameter.

24. The method of claim 23, wherein the test agent is a chemotherapeutic agent, a tyrosine kinase inhibitor (TKI), a VEGFR2/PDGFR-inhibiting tyrosine kinase inhibitor, or an infectious agent.

25. (canceled)

26. (canceled)

27. (canceled)

28. The method of claim 23, wherein assessing the test agent comprises assessing cardiotoxicity of the test agent.

29. The method of claim 23, wherein measuring the parameter comprises;
measuring a phenotype of interest, expression level of a gene of interest, or expression level of a protein of interest, or combinations thereof;
measuring fluorescence;
measuring sarcomere contractility or cell contractility;
measuring TEER resistance;
measuring expression levels of MYH7, MYH6, or both;
measuring expression levels of CDH5(CD144). PECAM1 (DC31), KDR (VEGFR2), NR2F2 (COUP-TFII), EFNB2, TEK (TIE2), MYH6, MYH7, ACTN2, or TNNT2 or combinations thereof;
measuring cell viability; or
obtaining calcium imaging of the iPSC-ECs or the iPSC-CCs, or both.

30. (canceled)

31. (canceled)

32. (canceled)

33. (canceled)

34. (canceled)

35. (canceled)

36. (canceled)

37. The method of claim 23, wherein contacting the test agent to the device or contacting the iPSC-CCs, iPSC-ECs, or both, with the test agent comprises:
culturing the iPSC-CCs, iPSC-ECs, or both, in the presence of liver-conditioned media;
further culturing the iPSC-CCs, iPSC-ECs, or both with an agent capable of activating Akt signaling; or
culturing the iPSC-CCs, iPSC-ECs, or both in the presence of culture media flowing through the device.

38. (canceled)

39. (canceled)

40. The method of claim 23, wherein the iPSC-ECs and iPSC-CCs are patient specific and the method models patient-specific parameters.

41. The method of claim 23, wherein
the iPSC-ECs are induced pluripotent stem cell derived-vascular endothelial cells (iPSC-vECs); and
the iPSC-CCs are induced pluripotent stem cell derived-cardiomyocytes (iPSC-CMs).

42. A method of producing the device of claim 1, comprising:
seeding (the iPSC-CCs on one surface of the membrane in the device; and seeding
(the iPSC-ECs on the other surface of the membrane in the device; OR
seeding (the iPSC-CCs in one chamber in the device; and seeding the iPSC-ECs in the other chamber in the device.

43. The method of claim 42, wherein
the iPSC-ECs are induced pluripotent stem cell derived-vascular endothelial cells (iPSC-vECs); and the iPSC-CCs are induced pluripotent stem cell derived-cardiomyocytes (iPSC-CMs), induced pluripotent stem cell derived-fibroblast cells (iPSC-FCs), or induced pluripotent stem cell derived-smooth muscle cells (iPSC-SMCs), OR
the iPSC-ECs are iPSC-vECs and the iPSC-CCs are iPSC-CMs.

44. (canceled)
